(19) United States
(12) Patent Application Publication
(10) Pub. No.: US 2026/0248430 A1
(43) Pub. Date: Aug. 27, 2026

(54) SYSTEM

(71) Applicant: SoftBank Group Corp., Tokyo (JP)

(72) Inventor: Takumi KUBO, Tokyo (JP)

(73) Assignee: SoftBank Group Corp., Tokyo (JP)

(21) Appl. No.: 19/535,010

(22) Filed: Feb. 10, 2026

(30) Foreign Application Priority Data
Feb. 21, 2025 (JP) 2025-027082

(51) Int. Cl. A61B 5/16; A61B 5/00; A61B 5/11; G06N 5/04; G06V 40/16; G10L 25/30; G10L 25/63; G16H 40/67; H04L 51/10

(52) U.S. Cl. CPC A61B 5/165; A61B 5/0077; A61B 5/1118; A61B 5/4803; A61B 5/7203; A61B 5/7267; A61B 5/7278; G06N 5/04; G16H 40/67; H04L 51/10; G06V 40/174; G10L 25/30; G10L 25/63

(57) ABSTRACT

The system according to the embodiment comprises a collection unit, an analysis unit, a provision unit, a determination unit, and a linkage unit. The collection unit collects data. The analysis unit analyzes data collected by the collection unit. The provision unit provides advice based on an analysis result obtained by the analysis unit. The determination unit instantly determines changes in chronic conditions. The determination unit instantly determines changes in health status. The linkage unit realizes a social media integration function.

### CROSS-REFERENCE TO RELATED APPLICATION

The present application claims priority to and incorporates by reference the entire contents of Japanese Patent Application No. 2025-027082 filed in Japan on Feb. 21, 2025.

### BACKGROUND OF THE INVENTION

### 1. Field of the Invention

The technology of this disclosure relates to a system.

### 2. Description of the Related Art

Japanese Patent Application Laid-open No. 2022-180282 discloses a persona chatbot control method executed by at least one processor, comprising: receiving a user utterance, adding the user utterance to a prompt containing instructions related to the character of the chatbot, encoding the prompt, inputting the encoded prompt into a language model, and generating a chatbot utterance in response to the user utterance.

In conventional technology, analysis of health data and instant determination of changes in chronic conditions have not been sufficiently performed, leaving room for improvement.

### SUMMARY OF THE INVENTION

The system according to the embodiment comprises a collection unit, an analysis unit, a provision unit, a determination unit, and a linkage unit. The collection unit collects data. The analysis unit analyzes data collected by the collection unit. The provision unit provides advice based on an analysis result obtained by the analysis unit. The determination unit instantly determines changes in chronic conditions. The determination unit instantly determines changes in health status. The linkage unit realizes a social media integration function.

The above and other objects, features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a conceptual diagram showing an example configuration of a data processing system according to the first embodiment;

FIG. 2 is a conceptual diagram showing an example of main functions of a data processing device and a smart device according to the first embodiment;

FIG. 3 is a conceptual diagram showing an example configuration of a data processing system according to the second embodiment;

FIG. 4 is a conceptual diagram showing an example of main functions of a data processing device and smart glasses according to the second embodiment;

FIG. 5 is a conceptual diagram showing an example configuration of a data processing system according to the third embodiment;

FIG. 6 is a conceptual diagram showing an example of main functions of a data processing device and a headset-type terminal according to the third embodiment;

FIG. 7 is a conceptual diagram showing an example configuration of a data processing system according to the fourth embodiment;

FIG. 8 is a conceptual diagram showing an example of main functions of a data processing device and a robot according to the fourth embodiment;

FIG. 9 shows an emotion map where multiple emotions are mapped; and

FIG. 10 shows an emotion map where multiple emotions are mapped.

### DETAILED DESCRIPTION OF THE PREFERRED EMBODIMENTS

Hereinafter, an example of an embodiment of the system related to the technology disclosed herein will be described with reference to the attached drawings.

First, the terminology used in the following description will be explained.

In the following embodiments, a processor denoted by a reference numeral (hereinafter simply referred to as “processor”) may be a single computing device or a combination of multiple computing devices. The processor may be a single type of computing device or a combination of multiple types of computing devices. Examples of computing devices include a CPU (Central Processing Unit), GPU (Graphics Processing Unit), GPGPU (General-Purpose computing on Graphics Processing Units), APU (Accelerated Processing Unit), or TPU (Tensor Processing Unit), among others.

In the following embodiments, a RAM (Random Access Memory) denoted by a reference numeral is a memory where information is temporarily stored and used as a work memory by the processor.

In the following embodiments, a storage denoted by a reference numeral is one or more non-volatile storage devices for storing various programs and parameters. Examples of non-volatile storage devices include flash memory (SSD (Solid State Drive)), magnetic disks (e.g., hard disks), or magnetic tapes, among others.

In the following embodiments, a communication I/F (Interface) denoted by a reference numeral is an interface including a communication processor and an antenna, among others. The communication I/F manages communication between multiple computers. Examples of communication standards applicable to the communication I/F include wireless communication standards such as 5G (5th Generation Mobile Communication System), Wi-Fi (registered trademark), or Bluetooth (registered trademark), among others.

In the following embodiments, “A and/or B” means “at least one of A and B.” In other words, “A and/or B” means it may be only A, only B, or a combination of A and B. Moreover, when expressing three or more items connected by “and/or,” the same concept as “A and/or B” applies.

### First Embodiment

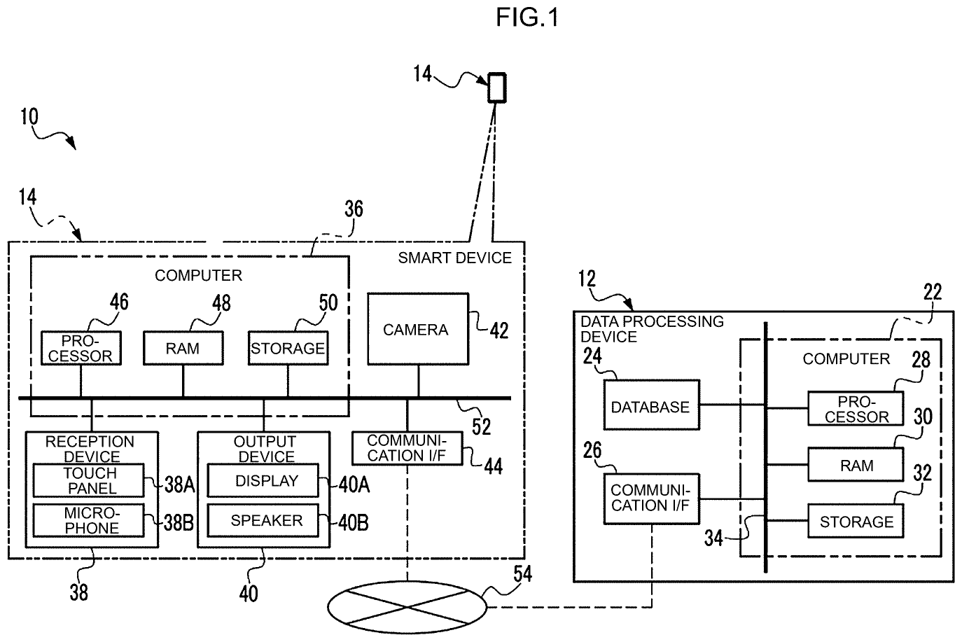

FIG. 1 shows an example configuration of a data processing system 10 according to the first embodiment.

As shown in FIG. 1, the data processing system 10 comprises a data processing device 12 and a smart device 14. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN (Wide Area Network) and/or a LAN (Local Area Network), among others.

The smart device 14 comprises a computer 36, a reception device 38, an output device 40, a camera 42, and a communication I/F 44. The computer 36 comprises a processor 46, RAM 48, and storage 50. The processor 46, RAM 48, and storage 50 are connected to a bus 52. The reception device 38, output device 40, and camera 42 are also connected to the bus 52.

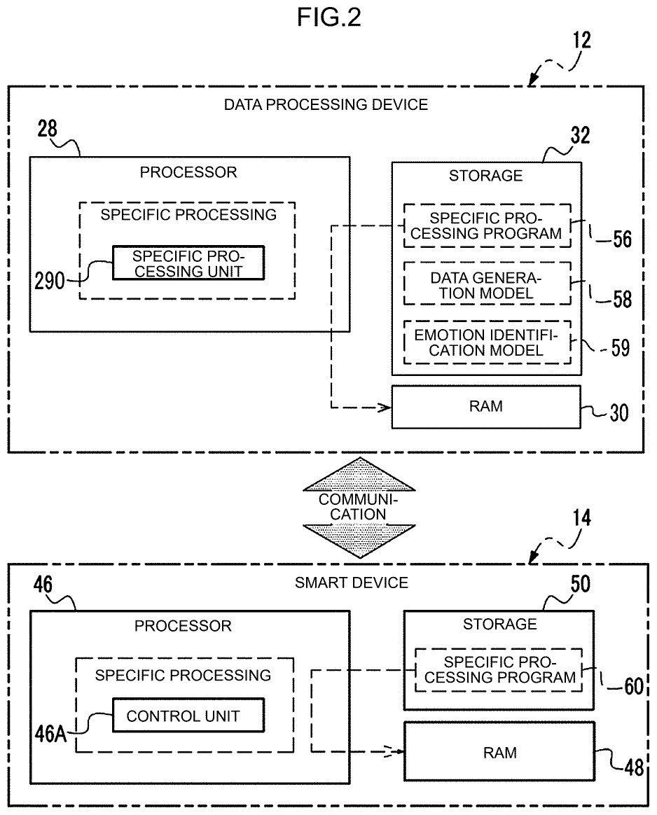

The reception device 38 comprises a touch panel 38A and a microphone 38B, among others, and accepts user input. The touch panel 38A accepts user input by detecting contact from an indicating object (e.g., a pen or finger). The microphone 38B accepts user input by detecting the user's voice. The control unit 46A sends data indicating user input accepted by the touch panel 38A and microphone 38B to the data processing device 12. The data processing device 12 has a specific processing unit 290 (see FIG. 2) that acquires data indicating user input.

The output device 40 comprises a display 40A and a speaker 40B, among others, and presents data to the user by outputting it in a perceptible form (e.g., audio and/or text). The display 40A displays visible information such as text and images according to instructions from the processor 46. The speaker 40B outputs audio according to instructions from the processor 46. The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors.

The communication I/F 44 is connected to the network 54. The communication I/F 44 and 26 manage the exchange of various information between the processor 46 and the processor 28 via the network 54.

FIG. 2 shows an example of the main functions of the data processing device 12 and the smart device 14.

As shown in FIG. 2, specific processing is performed in the data processing device 12 by the processor 28. The storage 32 stores a specific processing program 56. The specific processing program 56 is an example of a “program” related to the technology disclosed herein. The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the smart device 14, specific processing is performed by the processor 46. The storage 50 stores a specific processing program 60. The specific processing program 60 is used in conjunction with the specific processing program 56 by the data processing system 10. The processor 46 reads the specific processing program 60 from the storage 50 and executes it on the RAM 48. The specific processing is realized by the processor 46 operating as a control unit 46A according to the specific processing program 60 executed on the RAM 48. The smart device 14 may also have similar data generation models and emotion identification models as the data generation model 58 and emotion identification model 59, and perform the same processing as the specific processing unit 290 using these models.

Other devices besides the data processing device 12 may have the data generation model 58. For example, a server device (e.g., a generation server) may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.). Next, an example of processing by the data processing system 10 according to the first embodiment will be described.

### Example of the Embodiment

The health management system according to the embodiment of the present invention is a system that analyzes health data such as blood pressure, pulse, number of steps, body weight, and body temperature obtained from a wearable device using AI, and provides advice for improving health. This health management system transmits data obtained from the wearable device to AI in real time, and the AI analyzes the data to evaluate the user's health status. For example, if the blood pressure is high or the pulse is abnormally fast, the AI provides health advice based on that data. In addition, for users with chronic conditions or those deviating from the ideal health status, the AI instantly makes determinations. For example, if a diabetic patient shows a rapid fluctuation in blood glucose level, the AI detects the change and provides appropriate support. Furthermore, if necessary, help alerts are sent to medical facilities to prompt a rapid response. Additionally, the AI is equipped with a social media integration function, enabling users to connect with each other. For example, users with the same chronic condition can share information and support each other. In emergencies, alerts can be sent to nearby users to request help. In this way, the present invention supports health management by utilizing wearable devices and AI to monitor the user's health status in real time and provide appropriate advice and support. Moreover, the social media integration function allows people to connect and help each other, enabling users to live with greater peace of mind. As a result, the health management system can monitor the user's health status in real time and provide appropriate advice and support. Specifically, this health management system acquires multidimensional biometric information (e.g., blood pressure as a one-dimensional continuous value, pulse as a time-series array, number of steps as a daily cumulative value, body weight and body temperature as daily scalar values) from wearable devices in real time via Bluetooth Low Energy or Wi-Fi communication through the collection unit, and transmits these as structured tensor data to the analysis unit on the server along with time information and user ID. The analysis unit uses, for example, convolutional neural networks (CNN), recurrent neural networks (RNN), or Transformer-based time-series analysis models to perform anomaly detection and health status scoring on the input biometric data. Examples of AI input include: (1) time-series tensor of one day's blood pressure, pulse, number of steps, body weight, and body temperature (shape: 5×24×1); (2) recent one-hour pulse variation array (shape: 60×1); (3) user attribute vector (age, gender, history of illness flags, etc.). Examples of AI output include: (1) health risk score (continuous value from 0.0 to 1.0); (2) abnormal event labels (e.g., hypertension, tachycardia, hypothermia, etc.); (3) recommended actions (e.g., hydration, rest, recommendation to visit a medical institution, etc. as category labels). These outputs are used by the provision unit to determine the content of notifications to users and whether to issue emergency alerts through threshold judgment and rule-based branching. Furthermore, the determination unit analyzes the relationship between the user's history of chronic conditions and past abnormal events using AI, and, for example, in the case of a rapid change in blood glucose for a diabetic patient, instantly calls an automatic reporting API to a medical institution, thereby realizing autonomous determination and action beyond conventional simple monitoring. Regarding the social media integration function, the linkage unit clusters anonymized health status metadata among users using AI, automatically groups users with similar disease tendencies or lifestyles, and generates information sharing channels. For example, it is possible to send group notifications such as “Hypertension alerts are frequently occurring today” to user groups with a tendency toward hypertension. In emergencies, alerts are sent limited to nearby users by combining location information and health status, promoting rapid mutual assistance. This series of processes is not merely automation of human tasks, but an improvement of computer technology itself through high-dimensional data analysis, anomaly detection, autonomous determination, and optimization of user linkage by AI (e.g., improvement of real-time performance, reduction of false alarm rate, efficiency of communication and computational resources, enhancement of user experience). Application fields include management of lifestyle-related diseases, remote medical support, corporate health management, monitoring in nursing care settings, and condition management for athletes. Technical effects include personalized health management by AI, improved responsiveness in case of abnormalities, formation of autonomous support networks among users, and real-time analysis and utilization of large-scale, diverse health data that was difficult with conventional rule-based or manual monitoring.

The health management system according to the embodiment comprises a collection unit, an analysis unit, a provision unit, a determination unit, and a linkage unit. The collection unit collects data from wearable devices worn by the user. The collected data may include, for example, blood pressure, pulse, number of steps, body weight, and body temperature, but is not limited to these examples. The collection unit, for example, acquires data from the wearable device in real time and transmits it to AI. The analysis unit analyzes the data collected by the collection unit. The analysis unit, for example, uses AI to analyze the data and evaluate the user's health status. For example, if the blood pressure is high or the pulse is abnormally fast, the analysis unit provides health advice based on that data. The provision unit provides advice based on the analysis result obtained by the analysis unit. The provision unit, for example, uses AI to generate specific advice content and provides it to the user. Examples include dietary suggestions and exercise recommendations. The determination unit instantly determines changes in chronic conditions and changes in health status. The determination unit, for example, uses AI to detect changes in chronic conditions and changes in health status and provides appropriate support. For example, if a diabetic patient shows a rapid fluctuation in blood glucose level, the determination unit detects the change and issues a help alert to medical facilities. The linkage unit realizes a social media integration function. The linkage unit, for example, uses AI to enable information sharing among users, allowing them to support each other. For example, users with the same chronic condition can share information, and in emergencies, alerts can be sent to nearby users to request help. As a result, the health management system according to the embodiment can monitor the user's health status in real time and provide appropriate advice and support. Furthermore, the social media integration function allows people to connect and help each other, enabling users to live with greater peace of mind. Specifically, this health management system acquires multidimensional biometric information such as blood pressure (one-dimensional continuous value), pulse (time-series array), number of steps (daily cumulative value), body weight and body temperature (daily scalar values) from wearable devices in real time via Bluetooth Low Energy or Wi-Fi communication through the collection unit, and transmits these as structured tensor data (e.g., time-series tensor of 5×24×1) to the analysis unit on the server along with timestamps and user ID. The analysis unit uses convolutional neural networks (CNN), recurrent neural networks (RNN), or Transformer-based time-series analysis models to perform anomaly detection and health status scoring on the input biometric data. Examples of AI input include time-series tensor of one day's blood pressure, pulse, number of steps, body weight, and body temperature, recent one-hour pulse variation array, and user attribute vector (age, gender, history of illness flags, etc.). Examples of AI output include health risk score (continuous value from 0.0 to 1.0), abnormal event labels (hypertension, tachycardia, hypothermia, etc.), and recommended actions (e.g., hydration, rest, recommendation to visit a medical institution as category labels). These outputs are used by the provision unit to determine the content of notifications to users and whether to issue emergency alerts through threshold judgment and rule-based branching. The determination unit analyzes the relationship between the user's history of chronic conditions and past abnormal events using AI, and, for example, in the case of a rapid change in blood glucose for a diabetic patient, instantly calls an automatic reporting API to a medical institution, thereby realizing autonomous determination and action beyond conventional simple monitoring. The linkage unit clusters anonymized health status metadata among users using AI, automatically groups users with similar disease tendencies or lifestyles, and generates information sharing channels. For example, it is possible to send group notifications such as “Hypertension alerts are frequently occurring today” to user groups with a tendency toward hypertension. In emergencies, alerts are sent limited to nearby users by combining location information and health status, promoting rapid mutual assistance. This series of processes is not merely automation of human tasks, but an improvement of computer technology itself through high-dimensional data analysis, anomaly detection, autonomous determination, and optimization of user linkage by AI (improvement of real-time performance, reduction of false alarm rate, efficiency of communication and computational resources, enhancement of user experience). Application fields include management of lifestyle-related diseases, remote medical support, corporate health management, monitoring in nursing care settings, and condition management for athletes. Technical effects include personalized health management by AI, improved responsiveness in case of abnormalities, formation of autonomous support networks among users, and real-time analysis and utilization of large-scale, diverse health data that was difficult with conventional rule-based or manual monitoring.

The collection unit is equipped with a function to transmit data in real time. For example, the collection unit transmits data obtained from a wearable device to AI in real time. The specific time range for real-time transmission may include, for example, within a few seconds or within a few minutes, but is not limited to these examples. By transmitting data in real time, the user's health status can be instantly grasped. Some or all of the above-described processing in the collection unit may be performed using AI or without using AI. For example, the collection unit may transmit data obtained from the wearable device to AI, and the AI analyzes the data. Specifically, the collection unit incorporates Bluetooth Low Energy and Wi-Fi communication modules, and acquires pulse data every second (time-series array: 60×1), blood pressure values every five minutes (continuous value: 12×1), daily number of steps (scalar value), body weight and body temperature (scalar values) from the wearable device in real time. The acquired data is transmitted to the analysis unit on the server as structured tensors (e.g., 5×24×1) along with timestamps and user ID. Examples of AI input include: (1) recent one-minute pulse array (60×1); (2) time-series tensor of one day's blood pressure, pulse, number of steps, body weight, and body temperature; (3) user attribute vector (age, gender, history of illness flags, etc.). Examples of AI output include: (1) health risk score (continuous value from 0.0 to 1.0); (2) abnormal event labels (hypertension, tachycardia, etc.); (3) recommended actions (rest, hydration, etc.). These outputs are used by the provision unit and determination unit for threshold judgment and rule-based branching, determining whether to notify the user immediately or issue emergency alerts. Real-time transmission greatly improves early detection of health abnormalities and responsiveness compared to conventional batch processing systems, enabling qualitative improvement of user experience and rapid response in medical settings. Application fields include emergency medical support, real-time monitoring in sports settings, and monitoring in home care.

The analysis unit is equipped with a function to detect abnormal values. For example, the analysis unit analyzes the collected data using AI and detects abnormal values. Specific criteria and detection methods for abnormal values may include, for example, degree of deviation from the normal range or values exceeding specific thresholds, but are not limited to these examples. For example, the analysis unit detects abnormal values when blood pressure exceeds the normal range or when the pulse is abnormally fast. By detecting abnormal values, abnormalities in the user's health status can be discovered early. Some or all of the above-described processing in the analysis unit may be performed using AI or without using AI. For example, the analysis unit inputs the collected data to AI, and the AI detects abnormal values. Specifically, the analysis unit uses convolutional neural networks (CNN), recurrent neural networks (RNN), or Transformer-based time-series anomaly detection models to analyze multidimensional time-series data such as blood pressure, pulse, body temperature, and number of steps. Examples of AI input include: (1) time-series tensor of one day's blood pressure, pulse, number of steps, body weight, and body temperature (5×24×1); (2) recent one-hour pulse variation array (60×1); (3) user attribute vector (age, gender, history of illness flags, etc.). Examples of AI output include: (1) abnormal score (continuous value from 0.0 to 1.0); (2) abnormal event labels (hypertension, tachycardia, hypothermia, etc.); (3) structured data such as abnormal occurrence time and duration. These outputs are used by the provision unit and determination unit as triggers for threshold judgment and alert issuance. Anomaly detection algorithms may include reconstruction error-based detection using autoencoders or classification models using supervised learning. Unlike conventional simple threshold judgment, AI models realize highly accurate anomaly detection considering multivariate, time-series, and individual differences, resulting in technical effects such as reduction of false alarms and improvement of early detection rates. Application fields include early sign detection of chronic diseases, monitoring of overtraining in athletes, and detection of abnormal behavior in nursing care settings.

The provision unit is equipped with a function to generate specific advice content. For example, the provision unit generates specific advice content based on the analysis result using AI. Types and generation methods of specific advice content may include, for example, dietary suggestions and exercise recommendations, but are not limited to these examples. For example, the provision unit advises to reduce salt intake when blood pressure is high. The provision unit can also generate advice recommending walking in case of lack of exercise. By generating specific advice content, appropriate health advice can be provided to the user. Some or all of the above-described processing in the provision unit may be performed using AI or without using AI. For example, the provision unit inputs the analysis result to AI, and the AI generates specific advice content. Specifically, the provision unit inputs health risk scores and abnormal event labels output from the analysis unit, as well as user attribute vectors (age, gender, history of illness, etc.), to generative large language models or rule-based AI to generate personalized advice sentences. Examples of AI input include: (1) structured data such as “Blood pressure: 145/95, Pulse: 110, History: Hypertension”; (2) dataset such as “Steps: 3000, Goal: 8000, Age: 65”; (3) attribute vector such as “Weight: 80 kg, BMI: 28, Gender: Male”. Examples of AI output include: (1) natural language advice such as “Your blood pressure is high today. Please reduce salt intake and stay hydrated.”; (2) “You have not reached your step goal. We recommend a light walk in the evening.”; (3) “Your BMI is high. Please focus on a vegetable-based diet.” These outputs are immediately displayed on the user interface, and the notification method (push notification, email, etc.) is automatically selected according to urgency. AI-based advice generation, unlike conventional template-based notifications, realizes flexible expression and content optimization according to individual health status and history, resulting in technical effects such as promoting behavioral change and improving continuity of health management. Application fields include guidance for prevention of lifestyle-related diseases, sports coaching, support for corporate health management, and individualized care proposals in nursing care settings.

The determination unit is equipped with a function to issue help alerts to medical facilities. For example, the determination unit detects changes in chronic conditions and changes in health status using AI and issues help alerts to medical facilities. Specific content and methods of help alerts may include, for example, notification to emergency contacts or automatic reporting to medical institutions, but are not limited to these examples. For example, the determination unit issues a help alert to medical facilities when a diabetic patient shows a rapid fluctuation in blood glucose level. The determination unit can also notify emergency contacts when signs of a heart attack are detected. By issuing help alerts to medical facilities, rapid response in emergencies can be promoted. Some or all of the above-described processing in the determination unit may be performed using AI or without using AI. For example, the determination unit inputs changes in chronic conditions and changes in health status to AI, and the AI issues help alerts. Specifically, the determination unit inputs abnormal event labels (e.g., hyperglycemia, tachycardia, hypothermia, etc.) output from the analysis unit, health risk scores, user history of chronic conditions, and time-series data of past abnormal events to rule-based AI or decision tree models to determine urgency. Examples of AI input include: (1) time-series data such as “Blood glucose: 250→80 (rapid drop in 30 minutes), History: Diabetes”; (2) structured data such as “Pulse: 120, Chest pain flag: 1, Age: 70”; (3) dataset such as “Body temperature: 35.0, Decreased consciousness flag: 1”. Examples of AI output include: (1) action instruction data such as “Emergency alert: Call medical institution automatic reporting API”; (2) “Emergency contact SMS notification”; (3) “Push notification to user terminal”. These outputs are automatically sent to medical institutions or family terminals via communication modules. AI-based emergency determination, unlike conventional manual monitoring or simple threshold judgment, comprehensively considers multiple parameters, time-series variations, and individual history, resulting in technical effects such as reduction of false alarms and improved responsiveness in emergencies. Application fields include home medical care, monitoring of elderly living alone, accident response in sports settings, and emergency management for patients with chronic diseases.

The linkage unit is equipped with an information sharing function among users. For example, the linkage unit realizes information sharing among users using AI. Specific content and implementation methods of the information sharing function may include, for example, what information is shared, the scope and method of sharing, but are not limited to these examples. For example, the linkage unit enables users with the same chronic condition to share information and support each other. The linkage unit can also issue alerts to nearby users in emergencies to request help. By providing an information sharing function among users, users can support each other. Some or all of the above-described processing in the linkage unit may be performed using AI or without using AI. For example, the linkage unit inputs information among users to AI, and the AI realizes information sharing. Specifically, the linkage unit anonymizes and structures user health status metadata (e.g., disease tendencies, lifestyle habits, recent abnormal event history, location information, etc.), and uses clustering AI or graph neural networks to automatically generate user groups with similar tendencies. Examples of AI input include: (1) user attribute vector such as “Hypertension tendency: 1, Exercise habit: twice a week, Age: 60”; (2) array of recent one-week abnormal event history; (3) dataset such as “Location information: latitude and longitude, Current location: home”. Examples of AI output include: (1) group generation and notification instruction data such as “Generate information sharing channel for hypertension group”; (2) “Notification of frequent hypertension alerts today”; (3) “Issue emergency alert to nearby users”. These outputs are automatically reflected through user terminal apps or web interfaces. AI-based user linkage, unlike conventional manual grouping or bulletin board-type information sharing, realizes real-time, automatic, anonymized, and personalized linkage optimization, resulting in technical effects such as promotion of mutual assistance and improved efficiency of information sharing among users. Application fields include peer support for patients with chronic diseases, regional monitoring networks, group guidance for corporate health management, and condition sharing for sports teams.

The collection unit can estimate the user's emotion and adjust the data collection frequency based on the estimated emotion of the user. For example, the collection unit estimates the user's emotion using AI and adjusts the data collection frequency based on the estimated emotion. Specific methods and criteria for emotion estimation may include, for example, facial recognition, voice analysis, and self-reporting, but are not limited to these examples. For example, if the user is feeling stressed, the collection unit increases the data collection frequency to obtain more detailed health status. If the user is relaxed, the collection unit can decrease the data collection frequency to reduce the user's burden. Furthermore, if the user is exercising, the collection unit can increase the data collection frequency to record the effects of exercise in detail. By adjusting the data collection frequency according to the user's emotion, more detailed health status can be obtained. Emotion estimation is realized using, for example, an emotion engine or generative AI with an emotion estimation function. Generative AI may include text generation AI (e.g., LLM) or multimodal generative AI, but is not limited to these examples. Some or all of the above-described processing in the collection unit may be performed using AI or without using AI. For example, the collection unit inputs the user's emotion data to AI, and the AI adjusts the data collection frequency. Specifically, the collection unit acquires the user's facial images (RGB image: 224×224×3), voice waveform data (one-second sampling array: 16000×1), and self-report text (e.g., natural language sentences such as “I'm tired today”). The collection unit inputs these multimodal data to a multimodal emotion estimation AI that combines facial feature extraction by a pre-trained convolutional neural network (CNN), voice emotion recognition by a recurrent neural network (RNN), and text emotion classification by a large language model. Examples of AI input include: (1) “Facial image: smile, Voice: high tone, Text: I'm feeling good today”; (2) “Facial image: frown, Voice: low tone, Text: I'm stressed”; (3) “Facial image: expressionless, Voice: normal, Text: none”. Examples of AI output include: (1) emotion label (e.g., stress, relaxation, normal); (2) emotion intensity score (continuous value from 0.0 to 1.0); (3) recommended collection frequency (e.g., every 1 minute, every 5 minutes, every 30 minutes, etc.). The collection unit automatically adjusts the data acquisition interval based on the emotion label and intensity score output by AI using a collection frequency control module. For example, if the stress intensity is 0.8 or higher, the interval is set to every 1 minute; if in a relaxed state with 0.2 or lower, every 30 minutes. These outputs are reflected in the collection scheduler in real time, enabling data acquisition that instantly responds to changes in the user's condition. AI-based emotion estimation and collection frequency control, unlike conventional fixed-interval collection methods, realize dynamic data acquisition according to the user's psychological and physiological state, reducing unnecessary data accumulation and user burden while improving the early detection rate of abnormal signs. Application fields include stress management for patients with chronic diseases, mental health monitoring, condition management for athletes, and monitoring in home care. Technical effects include realization of personalized health monitoring and data efficiency that were difficult with conventional manual or simple rule-based methods, by combining AI-based multimodal emotion estimation and real-time collection frequency control.

The collection unit can analyze the user's past health data and select an appropriate collection timing. For example, the collection unit analyzes the user's past health data using AI and selects the optimal collection timing. Specific criteria and selection methods for appropriate collection timing may include, for example, timing based on analysis results of past data, but are not limited to these examples. For example, if the user's past data shows a tendency for high blood pressure in the morning, the collection unit increases the data collection frequency in the morning. If the user's past data shows a tendency for pulse to rise sharply after exercise, the collection unit increases the data collection frequency after exercise. Furthermore, if the user's past data shows a tendency for body temperature to fluctuate at night, the collection unit increases the data collection frequency at night. By analyzing the user's past health data, the optimal collection timing can be selected. Some or all of the above-described processing in the collection unit may be performed using AI or without using AI. For example, the collection unit inputs the user's past health data to AI, and the AI selects the appropriate collection timing. Specifically, the collection unit accumulates time-series tensor data for each user, such as blood pressure (24 times per day×30 days=720 points), pulse (every minute×24 hours×30 days), number of steps (once per day×30 days), body weight and body temperature (once per day×30 days) for the past month on the server. The collection unit inputs these multidimensional time-series data to an autoregressive recurrent neural network (RNN) or time-series clustering AI to extract features such as periodicity, abnormal tendencies, and peak occurrence times. Examples of AI input include: (1) time-series pattern data such as “Blood pressure: frequent high values at 7 a.m., stable at night”; (2) “Pulse: sharp rise for 30 minutes after exercise”; (3) “Body temperature: fluctuates by 0.5 degrees at night”. Examples of AI output include: (1) recommended collection timing (e.g., every 5 minutes from 6 to 8 a.m., every 30 minutes during the day); (2) event triggers (e.g., every 1 minute for 10 minutes after exercise detection); (3) instructions to strengthen night monitoring (e.g., every 10 minutes from 10 p.m. to 6 a.m.). The collection unit automatically sets the data acquisition interval based on the recommended timing output by AI using a collection scheduler. These outputs are dynamically optimized according to the user's lifestyle and health risks, and unlike conventional uniform schedule methods, result in technical effects such as prevention of missing important events and reduction of data redundancy. Application fields include early sign detection of lifestyle-related diseases, performance management for athletes, night monitoring of elderly at home, and corporate health management monitoring. Technical effects include simultaneous improvement of data utility and efficiency of communication and storage resources by introducing AI-based personalized collection timing control.

The collection unit can change the type of data to be collected based on the user's current activity status at the time of data collection. For example, the collection unit uses AI to grasp the user's current activity status at the time of data collection and changes the type of data to be collected. Specific methods and criteria for grasping current activity status may include, for example, exercising, resting, working, etc., but are not limited to these examples. For example, if the user is exercising, the collection unit prioritizes collection of heart rate and number of steps. If the user is resting, the collection unit can prioritize collection of body temperature and blood pressure. Furthermore, if the user is eating, the collection unit can prioritize collection of blood glucose and body weight. By changing the type of data to be collected based on the user's current activity status, more appropriate data can be collected. Some or all of the above-described processing in the collection unit may be performed using AI or without using AI. For example, the collection unit inputs the user's current activity status to AI, and the AI changes the type of data to be collected. Specifically, the collection unit acquires various activity context data in real time, such as accelerometer data (three-axis acceleration: 100 Hz×3), gyroscope data, GPS location information, user schedule information (e.g., calendar API integration), and voice commands or touch operation logs. The collection unit inputs these data to a convolutional neural network (CNN) or time-series classification AI to estimate activity labels such as “exercising,” “resting,” “working,” or “eating.” Examples of AI input include: (1) “Acceleration: large variation, GPS: moving, Calendar: exercise scheduled”→exercising; (2) “Acceleration: slight, Heart rate: stable, Calendar: lunch”→eating; (3) “Acceleration: almost zero, Body temperature: stable, Calendar: sleeping”→resting. Examples of AI output include: (1) activity labels (exercising, resting, working, eating, etc.); (2) recommended data types to collect (e.g., heart rate and number of steps during exercise, blood glucose and body weight during eating, body temperature and blood pressure during rest, etc.); (3) collection priority score (continuous value from 0.0 to 1.0). The collection unit automatically switches the target data to be collected based on the activity label and recommended data types output by AI using a sensor control module. These outputs optimize the content of data collection in response to changes in user activity, and unlike conventional uniform collection methods, result in technical effects such as suppression of unnecessary data acquisition and comprehensive acquisition of important data. Application fields include performance analysis in sports settings, understanding of daily rhythms in home care, monitoring of work safety in companies, and management of lifestyle-related diseases. Technical effects include realization of situation-adaptive health monitoring and data efficiency for each user by combining AI-based activity recognition and data type control.

The collection unit can estimate the user's emotion and determine the priority of data to be collected based on the estimated emotion of the user. For example, the collection unit estimates the user's emotion using AI and determines the priority of data to be collected based on the estimated emotion. Specific methods and criteria for emotion estimation may include, for example, facial recognition, voice analysis, and self-reporting, but are not limited to these examples. For example, if the user is feeling stressed, the collection unit prioritizes collection of heart rate and blood pressure. If the user is relaxed, the collection unit can prioritize collection of body temperature and body weight. Furthermore, if the user is exercising, the collection unit can prioritize collection of number of steps and heart rate. By determining the priority of data to be collected according to the user's emotion, more important data can be preferentially collected. Emotion estimation is realized using, for example, an emotion engine or generative AI with an emotion estimation function. Generative AI may include text generation AI (e.g., LLM) or multimodal generative AI, but is not limited to these examples. Some or all of the above-described processing in the collection unit may be performed using AI or without using AI. For example, the collection unit inputs the user's emotion data to AI, and the AI determines the priority of data to be collected. Specifically, the collection unit acquires multimodal emotion data such as the user's facial images (224×224×3), voice waveform (16000×1), and self-report text (natural language sentences), and inputs these to a multimodal emotion estimation AI (CNN+RNN+large language model). Examples of AI input include: (1) “Facial expression: frown, Voice: low tone, Text: feeling irritated”→stress; (2) “Facial expression: smile, Voice: normal, Text: feeling calm”→relaxation; (3) “Facial expression: serious, Voice: slightly high, Text: exercising”→exercising. Examples of AI output include: (1) emotion label (stress, relaxation, exercising, etc.); (2) data collection priority list (e.g., prioritize heart rate and blood pressure during stress, body temperature and body weight during relaxation, number of steps and heart rate during exercise); (3) priority score (continuous value from 0.0 to 1.0). The collection unit automatically optimizes the data collection order based on the priority list output by AI using a sensor control module. These outputs enable preferential acquisition of important data according to the user's psychological state and activity status, and unlike conventional uniform collection methods, result in technical effects such as early detection of abnormal signs and reduction of data redundancy. Application fields include mental health monitoring, management of chronic diseases, stress management for athletes, and monitoring in home care. Technical effects include realization of personalized health monitoring and data efficiency by combining AI-based emotion estimation and data priority control.

The collection unit can preferentially collect highly relevant data by considering the user's geographic location information at the time of data collection. For example, the collection unit considers the user's geographic location information using AI and preferentially collects highly relevant data. Specific methods for acquiring and using geographic location information may include, for example, GPS data and location information services, but are not limited to these examples. For example, if the user is at a high altitude, the collection unit prioritizes collection of oxygen saturation and heart rate. If the user is in an urban area, the collection unit can prioritize collection of number of steps and stress level. Furthermore, if the user is at home, the collection unit can prioritize collection of body temperature and body weight. By considering the user's geographic location information and preferentially collecting highly relevant data, more appropriate data can be collected. Some or all of the above-described processing in the collection unit may be performed using AI or without using AI. For example, the collection unit inputs the user's geographic location information to AI, and the AI preferentially collects highly relevant data. Specifically, the collection unit acquires latitude and longitude data from GPS sensors (e.g., 35.6895, 139.6917), altitude data, Wi-Fi/Bluetooth beacon information, and geographic features from map APIs (determination of urban area, high altitude, home, etc.) in real time. The collection unit inputs these geographic data to a geographic feature extraction AI (e.g., geographic information embedding model+rule-based AI) to estimate the environmental characteristics of the user's current location. Examples of AI input include: (1) “Latitude and longitude: altitude 2000 m, Surroundings: mountainous area”→high altitude; (2) “Latitude and longitude: urban center, Surroundings: office district”→urban area; (3) “Latitude and longitude: residential area, Wi-Fi: home SSID”→home. Examples of AI output include: (1) environment label (high altitude, urban area, home, etc.); (2) recommended data types to collect (oxygen saturation and heart rate at high altitude, number of steps and stress level in urban area, body temperature and body weight at home, etc.); (3) collection priority list. The collection unit automatically switches the target data to be collected based on the environment label and recommended data types output by AI using a sensor control module. These outputs enable preferential acquisition of data related to health risks in response to the user's geographic environment, and unlike conventional uniform collection methods, result in technical effects such as early detection of health risks due to environmental changes and data efficiency. Application fields include health management for high-altitude climbers, stress monitoring in urban areas, condition management for home care patients, and health risk monitoring for travelers. Technical effects include realization of environment-adaptive health monitoring and data efficiency by combining AI-based geographic information analysis and data priority control.

The collection unit can analyze the user's social media activity at the time of data collection and collect related data. For example, the collection unit analyzes the user's social media activity using AI and collects related data. Specific methods and criteria for analyzing social media activity may include, for example, post content and activity frequency, but are not limited to these examples. For example, if the user posts about feeling stressed on social media, the collection unit prioritizes collection of heart rate and blood pressure. If the user posts about exercise, the collection unit can prioritize collection of number of steps and heart rate. Furthermore, if the user posts about meals, the collection unit can prioritize collection of body weight and blood glucose. By analyzing the user's social media activity, related data can be collected. Some or all of the above-described processing in the collection unit may be performed using AI or without using AI. For example, the collection unit inputs the user's social media activity to AI, and the AI collects related data. Specifically, the collection unit automatically acquires various metadata via API from multiple social media platforms used by the user, such as post text (natural language sentences), post time, posting frequency, presence of attached images or videos, and number of reactions (likes, comments, etc.). The collection unit inputs these data to a pre-trained large language model or multimodal AI (e.g., text+image classification model) to perform sentiment analysis of post content (e.g., category classification such as stress, exercise, meal, relaxation), activity tendency scoring (e.g., number of posts per day, positive/negative ratio), and extraction of health-related events (e.g., key phrases such as “I'm tired today,” “I went running,” “I ate out”). Examples of AI input include: (1) post text such as “I'm tired from work today. Feeling stressed.”+post time+image (frown); (2) post text such as “Achieved 5 km morning run!”+GPS movement log; (3) post text such as “Had grilled meat for dinner. Maybe ate too much.”+meal image. Examples of AI output include: (1) emotion label (stress, exercise, meal, etc.); (2) health-related event tag (e.g., stress post, exercise post, meal post); (3) recommended data types to collect list (e.g., heart rate and blood pressure for stress posts, number of steps and heart rate for exercise posts, body weight and blood glucose for meal posts); (4) collection priority score (continuous value from 0.0 to 1.0). The collection unit automatically switches the target data to be collected in real time based on the event tag and recommended data types output by AI using a sensor control module. For example, if a stress post is detected, the collection frequency of heart rate and blood pressure is increased; for exercise posts, continuous acquisition of number of steps and heart rate is prioritized; for meal posts, acquisition of body weight and blood glucose is strengthened. By linking the user's online activity with biometric data collection, personalized health monitoring that instantly responds to psychological and behavioral changes is realized, unlike conventional simple periodic collection methods. AI-based social media analysis combines natural language processing, image recognition, and time-series analysis for high-dimensional data processing, enabling real-time analysis of large numbers of posts and cross-platform health risk detection that is difficult with manual work. Technical effects include optimization of data collection in response to psychological and behavioral changes, improved early detection rate of abnormal signs, reduction of data redundancy, and enhancement of user experience. Application fields include mental health monitoring, management of lifestyle-related diseases, SNS-linked condition management for athletes, monitoring in home care, and support for corporate health management.

The analysis unit can estimate the user's emotion and adjust the analysis algorithm based on the estimated emotion of the user. For example, the analysis unit estimates the user's emotion using AI and adjusts the analysis algorithm based on the estimated emotion. Specific methods and criteria for emotion estimation may include, for example, facial recognition, voice analysis, and self-reporting, but are not limited to these examples. For example, if the user is feeling stressed, the analysis unit applies an algorithm that prioritizes analysis of stress-related data. If the user is relaxed, the analysis unit can apply an algorithm that evaluates overall health status. Furthermore, if the user is exercising, the analysis unit can apply an algorithm that evaluates exercise effects. By adjusting the analysis algorithm according to the user's emotion, more appropriate analysis results can be obtained. Emotion estimation is realized using, for example, an emotion engine or generative AI with an emotion estimation function. Generative AI may include text generation AI (e.g., LLM) or multimodal generative AI, but is not limited to these examples. Some or all of the above-described processing in the analysis unit may be performed using AI or without using AI. For example, the analysis unit inputs the user's emotion data to AI, and the AI adjusts the analysis algorithm. Specifically, the analysis unit inputs the user's facial images (224×224×3 RGB images), voice waveform data (one-second sampling array: 16000×1), and self-report text (natural language sentences) to a multimodal emotion estimation AI, which combines facial feature extraction by a convolutional neural network (CNN), voice emotion recognition by a recurrent neural network (RNN), and text emotion classification by a large language model to output emotion labels (e.g., stress, relaxation, exercising, etc.) and emotion intensity scores (continuous value from 0.0 to 1.0). Examples of AI input include: (1) “Facial image: frown, Voice: low tone, Text: feeling irritated”; (2) “Facial image: smile, Voice: normal, Text: feeling calm”; (3) “Facial image: serious, Voice: slightly high, Text: exercising”. Examples of AI output include: (1) emotion label (stress, relaxation, exercising, etc.); (2) emotion intensity score (continuous value from 0.0 to 1.0); (3) recommended analysis algorithm ID (e.g., stress-specific analysis model for stress, overall health evaluation model for relaxation, exercise effect evaluation model for exercising, etc.). The analysis unit automatically switches the analysis pipeline based on the emotion label and recommended algorithm ID output by AI using an analysis algorithm selection module. For example, if the stress intensity is high, autonomic nervous balance and heart rate variability analysis are prioritized; in a relaxed state, comprehensive health scoring is performed; during exercise, calorie consumption and exercise load evaluation are emphasized. These outputs are used for algorithm branching control within the analysis unit and optimization of input content for subsequent advice generation units. AI-based emotion estimation and dynamic switching of analysis algorithms, unlike conventional fixed analysis flows, realize flexible data analysis that instantly responds to the user's psychological and physiological state, resulting in technical effects such as improved analysis accuracy, suppression of unnecessary computational resource consumption, and enhancement of user experience. Application fields include stress-induced anomaly detection for patients with chronic diseases, condition analysis for athletes, mental health monitoring, and personalized health evaluation in home care. Technical effects include simultaneous realization of personalized health data analysis and computational efficiency that were difficult with conventional manual or simple rule-based methods, by combining AI-based multimodal emotion estimation and analysis algorithm control.

The analysis unit can refer to past health data during analysis to improve the accuracy of abnormal value detection. For example, the analysis unit refers to past health data using AI during analysis to improve the accuracy of abnormal value detection. Specific methods and criteria for improving the accuracy of abnormal value detection may include, for example, methods for referring to past data and techniques for improving accuracy, but are not limited to these examples. For example, the analysis unit refers to the user's past blood pressure data to improve the accuracy of detecting abnormal blood pressure values. The analysis unit can also refer to the user's past pulse data to improve the accuracy of detecting abnormal pulse. Furthermore, the analysis unit can refer to the user's past body temperature data to improve the accuracy of detecting abnormal body temperature. By referring to past health data, the accuracy of abnormal value detection can be improved. Some or all of the above-described processing in the analysis unit may be performed using AI or without using AI. For example, the analysis unit inputs past health data to AI, and the AI improves the accuracy of abnormal value detection. Specifically, the analysis unit accumulates time-series tensor data for each user, such as blood pressure (24 times per day×30 days=720 points), pulse (every minute×24 hours×30 days), and body temperature (once per day×30 days) for the past month on the server, and inputs these multidimensional time-series data to an autoregressive recurrent neural network (RNN) or time-series clustering AI to extract features such as individual normal ranges, periodicity, abnormal tendencies, and peak occurrence times. Examples of AI input include: (1) time-series pattern data such as “Blood pressure: frequent high values at 7 a.m., stable at night”; (2) “Pulse: sharp rise for 30 minutes after exercise”; (3) “Body temperature: fluctuates by 0.5 degrees at night”. Examples of AI output include: (1) abnormal score (continuous value from 0.0 to 1.0); (2) abnormal event labels (hypertension, tachycardia, hypothermia, etc.); (3) structured data such as abnormal occurrence time and duration. The analysis unit inputs the abnormal score and event labels output by AI to a threshold judgment module, dynamically sets the normal range for each user, and performs abnormal judgment. For example, the upper limit of normal is set as the 95th percentile value from the blood pressure distribution over the past 30 days, and only values exceeding this are judged as abnormal. These outputs are linked to subsequent advice generation units and emergency alert issuance units. AI-based past data reference anomaly detection, unlike conventional uniform threshold judgment, realizes highly accurate anomaly detection considering individual differences, time-series variations, and periodicity, resulting in technical effects such as reduction of false alarms and improvement of early detection rates. Application fields include early sign detection of lifestyle-related diseases, performance management for athletes, night monitoring of elderly at home, and corporate health management monitoring. Technical effects include simultaneous improvement of data utility and reduction of false detection and missed detection by introducing AI-based personalized anomaly detection algorithms.

The analysis unit can apply different analysis methods for each data category during analysis. For example, the analysis unit applies different analysis methods for each data category using AI during analysis. Specific classification methods for data categories and analysis methods may include, for example, health data, behavioral data, and environmental data, but are not limited to these examples. For example, the analysis unit applies an analysis method that considers time-of-day variation to blood pressure data. The analysis unit can also apply an analysis method that considers differences between exercise and rest to pulse data. Furthermore, the analysis unit can apply an analysis method that considers diurnal variation to body temperature data. By applying different analysis methods for each data category, more accurate analysis results can be obtained. Some or all of the above-described processing in the analysis unit may be performed using AI or without using AI. For example, the analysis unit inputs different analysis methods for each data category to AI, and the AI performs the analysis. Specifically, the analysis unit acquires health data (blood pressure, pulse, body temperature, body weight, etc.), behavioral data (number of steps, exercise intensity, activity labels, etc.), and environmental data (room temperature, humidity, atmospheric pressure, geographic location information, etc.) as independent input tensors, and applies AI models optimized for each category. For example, time-series convolutional neural networks (CNN) or autoregressive RNNs are used for blood pressure data to extract time-of-day variation patterns; state transition models (e.g., hidden Markov models or LSTM) are applied to pulse data with exercise/rest labels; and diurnal rhythm analysis AI (e.g., Fourier transform+time-series clustering) is applied to body temperature data. Examples of AI input include: (1) time-series tensor of blood pressure (24 times per day×30 days); (2) pulse array with exercise/rest labels every minute; (3) scalar values of body temperature once per day×30 days; (4) activity label category array (exercise, rest, meal, etc.); (5) time-series tensor of environmental data (room temperature, humidity, atmospheric pressure, etc.). Examples of AI output include: (1) abnormal score for each category (e.g., blood pressure abnormal 0.8, pulse abnormal 0.2, body temperature abnormal 0.1); (2) event labels (e.g., hypertension, tachycardia, hypothermia, overactivity, etc.); (3) recommended actions for each category (e.g., salt restriction for blood pressure abnormality, rest for pulse abnormality, hydration for body temperature abnormality, etc.). The analysis unit provides category-optimized information to subsequent advice generation units and emergency alert issuance units based on the category-specific abnormal scores and event labels output by AI. By flexibly switching analysis methods according to the diversity and complexity of data, unlike conventional uniform analysis methods, technical effects such as improved analysis accuracy, reduction of false alarms, and personalized health evaluation for each user are realized. Application fields include management of lifestyle-related diseases, performance analysis for athletes, multidimensional health monitoring in home care, and support for corporate health management. Technical effects include simultaneous realization of high-precision analysis of multiple data types and improvement of user experience by introducing AI-based category-optimized analysis.

The analysis unit can estimate the user's emotion and adjust the display method of analysis results based on the estimated emotion of the user. For example, the analysis unit estimates the user's emotion using AI and adjusts the display method of analysis results based on the estimated emotion. Specific methods and criteria for emotion estimation may include, for example, facial recognition, voice analysis, and self-reporting, but are not limited to these examples. For example, if the user is feeling stressed, the analysis unit provides a simple and highly visible display method. If the user is relaxed, the analysis unit can provide a display method that includes detailed information. Furthermore, if the user is in a hurry, the analysis unit can provide a display method that highlights key points. By adjusting the display method of analysis results according to the user's emotion, a more appropriate display method can be provided. Emotion estimation is realized using, for example, an emotion engine or generative AI with an emotion estimation function. Generative AI may include text generation AI (e.g., LLM) or multimodal generative AI, but is not limited to these examples. Some or all of the above-described processing in the analysis unit may be performed using AI or without using AI. For example, the analysis unit inputs the user's emotion data to AI, and the AI adjusts the display method of analysis results. Specifically, the analysis unit acquires multimodal emotion data such as the user's facial images (224×224×3), voice waveform (16000×1), and self-report text (natural language sentences), and inputs these to an emotion estimation AI (CNN+RNN+large language model) to output emotion labels (e.g., stress, relaxation, in a hurry, etc.) and emotion intensity scores (continuous value from 0.0 to 1.0). Examples of AI input include: (1) “Facial expression: frown, Voice: low tone, Text: feeling irritated”→stress; (2) “Facial expression: smile, Voice: normal, Text: feeling calm”→relaxation; (3) “Facial expression: serious, Voice: fast speech, Text: in a hurry”→in a hurry. Examples of AI output include: (1) emotion label; (2) emotion intensity score; (3) recommended display mode (e.g., simple display for stress, detailed display for relaxation, key point display for in a hurry, etc.). The analysis unit automatically adjusts the display content, layout, color scheme, and amount of information based on the recommended display mode output by AI using a user interface control module. For example, in a stress state, graphs and numbers are minimized and only important abnormalities are highlighted; in a relaxed state, detailed time-series graphs and statistical information are additionally displayed; in a hurry, only key points are displayed in large font. These outputs realize information presentation that instantly responds to the user's psychological state and usage situation, and unlike conventional uniform UIs, prevent increased stress due to information overload and missing important information, resulting in technical effects such as improved user experience. Application fields include stress management for patients with chronic diseases, on-site responsive monitoring for athletes, UI optimization for elderly in home care, and corporate health management dashboards. Technical effects include simultaneous realization of personalized information presentation and improved user satisfaction by combining AI-based emotion estimation and dynamic UI control.

The analysis unit can determine the priority of analysis based on the data collection timing during analysis. For example, the analysis unit determines the priority of analysis based on the data collection timing using AI during analysis. Specific methods and criteria for determining the priority of analysis may include, for example, data collection timing and importance, but are not limited to these examples. For example, the analysis unit prioritizes analysis of morning data to predict the user's health status for the day. The analysis unit can also prioritize analysis of post-exercise data to evaluate the effects of exercise. Furthermore, the analysis unit can prioritize analysis of nighttime data to evaluate sleep quality. By determining the priority of analysis based on the data collection timing, more important data can be preferentially analyzed. Some or all of the above-described processing in the analysis unit may be performed using AI or without using AI. For example, the analysis unit inputs the data collection timing to AI, and the AI determines the priority of analysis. Specifically, the analysis unit acquires input tensors such as timestamps assigned to each data (e.g., 2024-06-01 07:00:00), activity labels (post-exercise, before sleep, nighttime, etc.), and user schedule information (calendar API integration), and inputs them to a time-series priority estimation AI (e.g., time-series classification model+rule-based priority determination module). Examples of AI input include: (1) “Blood pressure: 7 a.m., Activity: just after waking up”; (2) “Pulse: 10 minutes after exercise, Activity: running”; (3) “Body temperature: 11 p.m., Activity: before sleep”. Examples of AI output include: (1) analysis priority score (continuous value from 0.0 to 1.0); (2) recommended analysis order list (e.g., morning data→post-exercise data→nighttime data); (3) analysis purpose label (e.g., daytime prediction, exercise effect evaluation, sleep evaluation, etc.). The analysis unit automatically optimizes the data analysis order based on the priority score and recommended order list output by AI using an analysis scheduler. For example, morning blood pressure data is analyzed with the highest priority as it is directly related to daily health risk prediction; post-exercise data is immediately input to the exercise effect evaluation module; nighttime data is preferentially input to the sleep evaluation AI. These outputs realize analysis that instantly responds to the user's lifestyle and health risks, and unlike conventional uniform batch analysis methods, result in technical effects such as prevention of missing important events and improvement of real-time performance. Application fields include early sign detection of lifestyle-related diseases, performance management for athletes, night monitoring of elderly at home, and corporate health management monitoring. Technical effects include simultaneous improvement of data utility and efficiency of analysis resources by introducing AI-based time-series priority control.

The analysis unit can adjust the order of analysis based on the relevance of the data during analysis. For example, the analysis unit adjusts the order of analysis based on the relevance of the data using AI during analysis. Specific methods for determining data relevance and adjusting the order of analysis may include, for example, correlation and causality, but are not limited to these examples. For example, the analysis unit analyzes blood pressure and pulse data together to evaluate cardiovascular health. The analysis unit can also analyze body temperature and body weight data together to evaluate metabolic health. Furthermore, the analysis unit can analyze number of steps and body weight data together to evaluate the effects of exercise. By adjusting the order of analysis based on the relevance of the data, more accurate analysis results can be obtained. Some or all of the above-described processing in the analysis unit may be performed using AI or without using AI. For example, the analysis unit inputs data relevance to AI, and the AI adjusts the order of analysis. Specifically, the analysis unit acquires multidimensional time-series data such as blood pressure, pulse, body temperature, body weight, and number of steps as structured tensors (e.g., 5×24×1), and inputs them to a correlation analysis AI (e.g., multivariate regression model, graph neural network, causal inference AI, etc.) to calculate correlation coefficients, causality scores, and importance indicators among the data. Examples of AI input include: (1) simultaneous time-series data of blood pressure and pulse; (2) one-month array of body temperature and body weight; (3) composite dataset of number of steps, body weight, and exercise intensity. Examples of AI output include: (1) relevance score matrix (e.g., blood pressure-pulse 0.85, body temperature-body weight 0.65, etc.); (2) recommended analysis order list (e.g., blood pressure→pulse→body weight→body temperature→number of steps); (3) analysis group label (e.g., cardiovascular system, metabolic system, exercise system, etc.). The analysis unit automatically optimizes the data analysis order and grouping based on the relevance score and recommended order list output by AI using an analysis pipeline control module. For example, if the correlation between blood pressure and pulse is high, simultaneous analysis is prioritized; if the relevance between body temperature and body weight is strong, they are input together to the metabolism evaluation module. These outputs contribute to comprehensive health evaluation and improved accuracy of anomaly detection using multiple data, and unlike conventional single-data sequential analysis methods, result in technical effects such as high-precision analysis considering interactions among data. Application fields include cardiovascular disease risk evaluation, detection of metabolic abnormalities, performance analysis for athletes, and multidimensional health monitoring in home care. Technical effects include simultaneous realization of high-precision analysis of multiple data types and improvement of user experience by introducing AI-based relevance analysis and order optimization.

The provision unit is capable of estimating the user's emotion and adjusting the method of expressing advice based on the estimated emotion of the user. For example, the provision unit uses AI to estimate the user's emotion and adjusts the method of expressing advice according to the estimated emotion. Specific methods and criteria for emotion estimation may include facial recognition, voice analysis, self-reporting, and the like, but are not limited thereto. For instance, when the user is feeling stressed, the provision unit provides advice in gentle language. When the user is relaxed, the provision unit can provide detailed advice. Furthermore, when the user is in a hurry, the provision unit can provide concise advice focusing on key points. By adjusting the method of expressing advice according to the user's emotion, more appropriate advice can be provided. Emotion estimation is realized, for example, by using an emotion engine or a generative AI as an emotion estimation function. The generative AI may be a text generation AI (e.g., LLM) or a multimodal generative AI, but is not limited thereto. Some or all of the above-described processing in the provision unit may be performed using AI or without using AI. For example, the provision unit inputs the user's emotion data to AI, and the AI adjusts the method of expressing advice. Specifically, the provision unit inputs the user's facial image (224×224×3 RGB image), voice waveform data (1-second sampling array: 16000×1), and self-reported text (natural language sentence) to a multimodal emotion estimation AI, which combines facial feature extraction by a convolutional neural network (CNN), voice emotion recognition by a recurrent neural network (RNN), and text emotion classification by a large language model to output emotion labels (e.g., stress, relaxation, hurry, etc.) and emotion intensity scores (0.0-1.0). Examples of AI input include: (1) “Facial image: frown, voice: low tone, text: feeling irritated”; (2) “Facial image: smile, voice: normal, text: feeling calm”; (3) “Facial image: serious, voice: fast, text: in a hurry”. Examples of AI output include: (1) emotion labels (stress, relaxation, hurry, etc.); (2) emotion intensity scores (0.0-1.0); (3) recommended expression modes (e.g., gentle expression for stress, detailed expression for relaxation, concise expression for hurry, etc.). Based on the recommended expression mode output by the AI, the advice generation module automatically adjusts the tone, style, amount of information, and emphasis points of the advice sentence using a natural language generation AI (large language model) or a rule-based generation engine. For example, in a stress state, a gentle tone such as “Take it easy and rest slowly” is selected; in a relaxed state, a detailed explanation such as “Your health condition today is good. Please continue to maintain a balanced diet and moderate exercise” is added; and in a hurry, only the key points such as “Don't forget to hydrate” are presented concisely. These outputs are immediately displayed as app screens or notification messages via the user interface control module. Emotion estimation and advice expression control by AI, unlike conventional uniform template notifications, realize personalized information presentation that responds to the user's psychological state and usage situation, thereby preventing increased stress due to information overload and missing important information, and providing the technical effect of improving user experience. Application fields include stress management for chronic disease patients, on-site advice for athletes, UI optimization for elderly home care, and corporate health management dashboards. The technical effect is that the combination of emotion estimation and dynamic advice expression control by AI enables simultaneous realization of personalized health support and improved user satisfaction.

The provision unit is capable of adjusting the level of detail of advice based on the importance of health data when providing advice. For example, the provision unit uses AI to adjust the level of detail of advice according to the importance of health data at the time of advice provision. Specific methods and criteria for adjusting the level of detail of advice may include the depth and specificity of information, among others, but are not limited thereto. For instance, when blood pressure is high, the provision unit provides detailed advice. When body temperature is within the normal range, the provision unit can provide concise advice. Furthermore, when the number of steps does not reach the target, the provision unit can provide specific exercise advice. By adjusting the level of detail of advice based on the importance of health data, more appropriate advice can be provided. Some or all of the above-described processing in the provision unit may be performed using AI or without using AI. For example, the provision unit inputs the importance of health data to AI, and the AI adjusts the level of detail of advice. Specifically, the provision unit inputs the health risk score (continuous value from 0.0 to 1.0) output from the analysis unit, abnormal event labels (e.g., hypertension, tachycardia, hypothermia, etc.), and user attribute vectors (age, gender, medical history, etc.) to an advice detail level control AI (e.g., rule-based +large language model). Examples of AI input include: (1) “Blood pressure: 150/100, risk score: 0.9, medical history: hypertension”; (2) “Body temperature: 36.5, risk score: 0.1, medical history: none”; (3) “Steps: 4000, target: 8000, risk score: 0.5”. Examples of AI output include: (1) recommended detail level (e.g., high, medium, low); (2) advice sentence generation parameters (e.g., amount of detailed explanation, number of specific suggestions); (3) advice category (e.g., urgent, normal, reference, etc.). Based on the detail level output by the AI, the natural language generation AI or rule-based generation engine automatically adjusts the amount of information, specificity, and depth of explanation in the advice sentence. For example, when the risk score is high, a detailed explanation and multiple specific suggestions such as “Your blood pressure is very high. Please reduce salt intake, rest, and consult a medical institution if necessary” are presented; when the risk is low, concise advice such as “Your body temperature is within the normal range. Please continue to manage your health” is generated; and when the number of steps does not reach the target, specific behavioral suggestions such as “You have not reached your target steps today. Add a light walk in the evening” are added. These outputs are immediately displayed as app screens or notification messages on the user's device, realizing optimal information provision according to the user's health status and risk. Detail level control by AI, unlike conventional uniform template notifications, enables flexible information presentation according to health risk and importance, and provides technical effects such as promoting behavioral change, preventing misunderstandings, and supporting rapid response in medical settings. Application fields include lifestyle disease management, performance guidance for athletes, abnormal response in home care, and corporate health management support. The technical effect is that the introduction of risk-based detail level control by AI enables simultaneous realization of personalized health support and prevention of information overload and information deficiency.

The provision unit is capable of applying different advice algorithms according to the category of health data when providing advice. For example, the provision unit uses AI to apply different advice algorithms according to the category of health data at the time of advice provision. Specific types and application methods of advice algorithms may include machine learning algorithms, rule-based algorithms, and the like, but are not limited thereto. For instance, the provision unit applies advice algorithms related to diet and exercise to blood pressure data. The provision unit can also apply advice algorithms related to dietary restrictions and exercise to body weight data. Furthermore, the provision unit can apply advice algorithms related to rest and hydration to body temperature data. By applying different advice algorithms according to the category of health data, more appropriate advice can be provided. Some or all of the above-described processing in the provision unit may be performed using AI or without using AI. For example, the provision unit inputs the category of health data to AI, and the AI applies the advice algorithm. Specifically, the provision unit classifies health data (blood pressure, body weight, body temperature, pulse, steps, etc.) output from the analysis unit by category and inputs them to category-specific advice AI (e.g., rule-based AI for blood pressure, machine learning model for body weight, generative AI for body temperature, etc.). Examples of AI input include: (1) blood pressure data (time-series tensor: 24 times per day×30 days); (2) body weight data (scalar value: once per day×30 days); (3) body temperature data (scalar value: once per day×30 days); (4) pulse data (labeled array for exercise/rest); (5) step data (daily cumulative value). Examples of AI output include: (1) category-specific recommended actions (e.g., salt restriction and exercise recommendation for blood pressure abnormality, dietary restriction and exercise suggestion for body weight increase, rest and hydration for body temperature decrease, etc.); (2) advice sentence generation parameters (e.g., explanation amount and tone for each category); (3) advice priority list. Based on the category-specific recommended actions output by the AI, the natural language generation AI or rule-based generation engine generates advice sentences optimized for each category. For example, for blood pressure data: “Your blood pressure is high. Please reduce salt intake and maintain moderate exercise”; for body weight data: “Your body weight is increasing. Review your diet and add walking”; for body temperature data: “Your body temperature is decreasing. Please take sufficient rest and hydrate” are automatically generated. These outputs are immediately displayed as app screens or notification messages on the user's device, realizing optimal information provision according to health risks and situations for each category. Category-optimized advice by AI, unlike conventional uniform template notifications, enables flexible information presentation reflecting expert knowledge and individual differences for each data type, and provides technical effects such as promoting behavioral change and improving continuity of health management. Application fields include lifestyle disease management, performance guidance for athletes, multifaceted health support in home care, and corporate health management support. The technical effect is that the introduction of category-optimized advice by AI enables simultaneous realization of high-precision support for multiple data types and improvement of user experience.

The provision unit is capable of estimating the user's emotion and adjusting the length of advice based on the estimated emotion of the user. For example, the provision unit uses AI to estimate the user's emotion and adjusts the length of advice according to the estimated emotion. Specific methods and criteria for emotion estimation may include facial recognition, voice analysis, self-reporting, and the like, but are not limited thereto. For instance, when the user is feeling stressed, the provision unit provides short and concise advice focusing on key points. When the user is relaxed, the provision unit can provide detailed advice. Furthermore, when the user is in a hurry, the provision unit can provide concise and prompt advice. By adjusting the length of advice according to the user's emotion, more appropriate advice can be provided. Emotion estimation is realized, for example, by using an emotion engine or a generative AI as an emotion estimation function. The generative AI may be a text generation AI (e.g., LLM) or a multimodal generative AI, but is not limited thereto. Some or all of the above-described processing in the provision unit may be performed using AI or without using AI. For example, the provision unit inputs the user's emotion data to AI, and the AI adjusts the length of advice. Specifically, the provision unit inputs multimodal emotion data such as the user's facial image (224×224×3), voice waveform (16000×1), and self-reported text (natural language sentence) to an emotion estimation AI (CNN+RNN+large language model), which outputs emotion labels (e.g., stress, relaxation, hurry, etc.) and emotion intensity scores (0.0-1.0). Examples of AI input include: (1) “Facial: frown, voice: low tone, text: feeling irritated”→stress; (2) “Facial: smile, voice: normal, text: feeling calm”→relaxation; (3) “Facial: serious, voice: fast, text: in a hurry”→hurry. Examples of AI output include: (1) emotion label; (2) emotion intensity score; (3) recommended advice length (e.g., short sentence for stress, long sentence for relaxation, key points only for hurry, etc.). Based on the recommended advice length output by the AI, the natural language generation AI or rule-based generation engine automatically adjusts the sentence length, amount of information, and depth of explanation in the advice sentence. For example, in a stress state, a short sentence such as “Take a rest without overdoing it” is generated; in a relaxed state, a detailed explanation such as “Your health condition today is good. Please continue a balanced diet and moderate exercise” is added; and in a hurry, only the key points such as “Don't forget to hydrate” are presented concisely. These outputs are immediately displayed as app screens or notification messages on the user's device, realizing optimal information provision that responds to the user's psychological state and usage situation. Advice length control by AI, unlike conventional uniform template notifications, enables flexible information presentation according to the user's situation and needs, and provides technical effects such as preventing increased stress due to information overload and missing important information, and improving user experience. Application fields include stress management for chronic disease patients, on-site advice for athletes, UI optimization for elderly home care, and corporate health management dashboards. The technical effect is that the combination of emotion estimation and dynamic advice length control by AI enables simultaneous realization of personalized health support and improved user satisfaction.

The provision unit is capable of determining the priority of advice based on the timing of health data collection when providing advice. For example, the provision unit uses AI to determine the priority of advice according to the timing of health data collection at the time of advice provision. Specific methods and criteria for determining the priority of advice may include the timing and importance of health data collection, among others, but are not limited thereto. For instance, the provision unit prioritizes advice related to morning routines based on morning data. The provision unit can also prioritize advice related to post-exercise care based on post-exercise data. Furthermore, the provision unit can prioritize advice related to sleep based on nighttime data. By determining the priority of advice based on the timing of health data collection, more appropriate advice can be provided. Some or all of the above-described processing in the provision unit may be performed using AI or without using AI. For example, the provision unit inputs the timing of health data collection to AI, and the AI determines the priority of advice. Specifically, the provision unit obtains input tensors such as timestamps attached to health data received from the analysis unit (e.g., 2024-06-01 07:00:00), activity labels (e.g., post-exercise, before bedtime, nighttime), and user schedule information (calendar API integration), and inputs them to a time-series priority estimation AI (e.g., time-series classification model+rule-based priority determination module). Examples of AI input include: (1) “Blood pressure: 7 a.m., activity: immediately after waking”; (2) “Pulse: 10 minutes after exercise, activity: running”; (3) “Body temperature: 11 p.m., activity: before bedtime”. Examples of AI output include: (1) advice priority score (0.0-1.0); (2) recommended advice order list (e.g., morning data→post-exercise data→nighttime data); (3) advice purpose label (e.g., daytime prediction, exercise care, sleep improvement, etc.). Based on the priority score and recommended order list output by the AI, the advice generation module automatically optimizes the generation order and notification timing of advice sentences. For example, advice based on morning blood pressure data is notified with the highest priority as it is directly related to daily health risk prediction; post-exercise data is used to immediately generate exercise care suggestions; and nighttime data is presented as sleep improvement advice tailored to nighttime. These outputs are immediately displayed as app screens or notification messages on the user's device, realizing optimal information provision that responds to the user's daily rhythm and health risks. Time-series priority control by AI, unlike conventional uniform template notifications, enables flexible information presentation according to the timing of data collection and important events, and provides technical effects such as promoting behavioral change, preventing missing important information, and supporting rapid response in medical settings. Application fields include lifestyle disease management, performance guidance for athletes, nighttime monitoring in home care, and corporate health management support. The technical effect is that the introduction of time-series priority control by AI enables simultaneous realization of personalized health support, prevention of information overload and information deficiency, and efficient use of analysis resources.

The provision unit is capable of adjusting the order of advice based on the relevance of health data when providing advice. For example, the provision unit uses AI to adjust the order of advice according to the relevance of health data at the time of advice provision. Specific methods and criteria for adjusting the order of advice may include the relevance and importance of health data, among others, but are not limited thereto. For instance, the provision unit associates blood pressure and pulse data to prioritize advice related to cardiovascular health. The provision unit can also associate body temperature and body weight data to prioritize advice related to metabolic health. Furthermore, the provision unit can associate step count and body weight data to prioritize advice related to exercise effects. By adjusting the order of advice based on the relevance of health data, more appropriate advice can be provided. Some or all of the above-described processing in the provision unit may be performed using AI or without using AI. For example, the provision unit inputs the relevance of health data to AI, and the AI adjusts the order of advice. Specifically, the provision unit obtains multidimensional time-series data such as blood pressure, pulse, body temperature, body weight, and step count as structured tensors (e.g., 5×24×1), and inputs them to a correlation analysis AI (e.g., multivariate regression model, graph neural network, causal inference AI, etc.) to calculate correlation coefficients, causal relationship scores, and importance indicators among the data. Examples of AI input include: (1) simultaneous time-series data of blood pressure and pulse; (2) one-month array of body temperature and body weight; (3) composite data set of step count, body weight, and exercise intensity. Examples of AI output include: (1) relevance score matrix (e.g., blood pressure-pulse 0.85, body temperature-body weight 0.65, etc.); (2) recommended advice order list (e.g., cardiovascular system→metabolic system→exercise system); (3) advice group labels (e.g., cardiovascular health, metabolic health, exercise effect, etc.). Based on the relevance scores and recommended order list output by the AI, the advice generation module automatically optimizes the generation order and notification content of advice sentences. For example, when the correlation between blood pressure and pulse is high, cardiovascular health advice is presented with the highest priority; when the relevance between body temperature and body weight is strong, metabolic health advice is notified together; and when the relevance between step count and body weight is high, exercise effect advice is emphasized. These outputs are immediately displayed as app screens or notification messages on the user's device, contributing to comprehensive health evaluation and improved abnormality detection accuracy for multiple data. Correlation analysis and order optimization by AI, unlike conventional sequential notification methods for single data, provide the technical effect of high-precision advice that considers interactions among data. Application fields include cardiovascular disease risk assessment, metabolic abnormality management, performance guidance for athletes, and multifaceted health support in home care. The technical effect is that the introduction of correlation analysis and advice order optimization by AI enables simultaneous realization of high-precision support for multiple data types and improvement of user experience.

The determination unit is capable of estimating the user's emotion and adjusting the determination criteria based on the estimated emotion of the user. For example, the determination unit uses AI to estimate the user's emotion and adjusts the determination criteria according to the estimated emotion. Specific methods and criteria for emotion estimation may include facial recognition, voice analysis, self-reporting, and the like, but are not limited thereto. For instance, when the user is feeling stressed, the determination unit adjusts the determination criteria to emphasize stress-related data. When the user is relaxed, the determination unit can adjust the criteria to emphasize overall health status. Furthermore, when the user is exercising, the determination unit can adjust the criteria to emphasize exercise effects. By adjusting the determination criteria according to the user's emotion, more appropriate determinations can be made. Emotion estimation is realized, for example, by using an emotion engine or a generative AI as an emotion estimation function. The generative AI may be a text generation AI (e.g., LLM) or a multimodal generative AI, but is not limited thereto. Some or all of the above-described processing in the determination unit may be performed using AI or without using AI. For example, the determination unit inputs the user's emotion data to AI, and the AI adjusts the determination criteria. Specifically, the determination unit inputs the user's facial image (224×224×3 RGB image), voice waveform data (1-second sampling array: 16000×1), and self-reported text (natural language sentence) to a multimodal emotion estimation AI, which combines facial feature extraction by a convolutional neural network (CNN), voice emotion recognition by a recurrent neural network (RNN), and text emotion classification by a large language model to output emotion labels (e.g., stress, relaxation, exercising, etc.) and emotion intensity scores (0.0-1.0). Examples of AI input include: (1) “Facial image: frown, voice: low tone, text: feeling irritated”; (2) “Facial image: smile, voice: normal, text: feeling calm”; (3) “Facial image: serious, voice: slightly high, text: exercising”. Examples of AI output include: (1) emotion label; (2) emotion intensity score; (3) recommended determination criteria set (e.g., emphasize stress-related indicators during stress, emphasize comprehensive health indicators during relaxation, emphasize exercise effect indicators during exercise, etc.). Based on the recommended determination criteria set output by the AI, the determination criteria control module automatically adjusts the weighting of abnormality determination and urgency evaluation. For example, when stress intensity is high, heart rate variability and autonomic balance indicators are emphasized; during relaxation, overall health scores are emphasized; and during exercise, calorie consumption and exercise load indicators are prioritized. These outputs are linked to subsequent alert transmission and advice generation units, realizing optimal determination that responds to the user's psychological and physiological state. Emotion estimation and dynamic determination criteria control by AI, unlike conventional fixed determination flows, enable flexible determination according to the user's situation, and provide technical effects such as reducing false positives and improving user experience. Application fields include stress-induced abnormality detection for chronic disease patients, condition evaluation for athletes, mental health monitoring, and personalized health management in home care. The technical effect is that the combination of emotion estimation and determination criteria control by AI enables simultaneous realization of personalized health determination and computational efficiency.

The determination unit is capable of referring to past health data at the time of determination to improve the accuracy of determination. For example, the determination unit uses AI to refer to past health data at the time of determination and improve the accuracy of determination. Specific methods and criteria for improving determination accuracy may include methods for referring to past data and techniques for improving accuracy, among others, but are not limited thereto. For instance, the determination unit refers to the user's past blood pressure data to improve the accuracy of detecting abnormal blood pressure values. The determination unit can also refer to the user's past pulse data to improve the accuracy of detecting abnormal pulse. Furthermore, the determination unit can refer to the user's past body temperature data to improve the accuracy of detecting abnormal body temperature. By referring to past health data, the accuracy of determination can be improved. Some or all of the above-described processing in the determination unit may be performed using AI or without using AI. For example, the determination unit inputs past health data to AI, and the AI improves the accuracy of determination. Specifically, the determination unit accumulates time-series tensor data for each user, such as past one month of blood pressure (24 times per day×30 days=720 points), pulse (every minute×24 hours×30 days), and body temperature (once per day×30 days) on the server, and inputs these multidimensional time-series data to an autoregressive recurrent neural network (RNN) or time-series clustering AI to extract features such as individual normal ranges, periodicity, abnormal trends, and peak occurrence times. Examples of AI input include: (1) “Blood pressure: frequent high values at 7 a.m., stable at night”; (2) “Pulse: rapid increase for 30 minutes after exercise”; (3) “Body temperature: 0.5° C. fluctuation at night”. Examples of AI output include: (1) abnormality score (continuous value from 0.0 to 1.0); (2) abnormal event label (hypertension, tachycardia, hypothermia, etc.); (3) structured data such as abnormal occurrence time and duration. The determination unit inputs the abnormality score and event label output by the AI to a threshold determination module, dynamically sets the normal range for each user, and performs abnormality determination. For example, the 95th percentile value of the blood pressure distribution over the past 30 days is set as the normal upper limit, and only values exceeding this are determined as abnormal. These outputs are linked to subsequent advice generation units and emergency alert transmission units. Past data reference-type abnormality determination by AI, unlike conventional uniform threshold determination, realizes high-precision abnormality detection that considers individual differences, time-series fluctuations, and periodicity, and provides technical effects such as reducing false positives and improving early detection rates. Application fields include early sign detection of lifestyle diseases, performance management for athletes, nighttime monitoring for elderly at home, and corporate health management monitoring. The technical effect is that the introduction of individually optimized abnormality determination algorithms by AI enables simultaneous realization of improved data utility and reduction of false detection and oversight.

The determination unit is capable of applying different determination methods for each category of health data at the time of determination. For example, the determination unit uses AI to apply different determination methods for each category of health data at the time of determination. Specific types and application methods of determination methods may include rule-based determination, machine learning algorithms, and the like, but are not limited thereto. For instance, the determination unit applies determination methods that consider time-of-day fluctuations to blood pressure data. The determination unit can also apply determination methods that consider differences between exercise and rest to pulse data. Furthermore, the determination unit can apply determination methods that consider diurnal fluctuations to body temperature data. By applying different determination methods for each category of health data, more accurate determinations can be made. Some or all of the above-described processing in the determination unit may be performed using AI or without using AI. For example, the determination unit inputs the category of health data to AI, and the AI applies the determination method. Specifically, the determination unit obtains health data (blood pressure, pulse, body temperature, body weight, etc.), behavioral data (step count, exercise intensity, activity labels, etc.), and environmental data (room temperature, humidity, atmospheric pressure, geographic location information, etc.) as independent input tensors, and applies AI models optimized for each category. For example, time-series convolutional neural networks (CNN) or autoregressive RNNs are used for blood pressure data to extract time-of-day fluctuation patterns; state transition models (e.g., hidden Markov models or LSTM) are applied to pulse data with exercise/rest labels; and diurnal rhythm analysis AI (e.g., Fourier transform+time-series clustering) is applied to body temperature data. Examples of AI input include: (1) blood pressure: time-series tensor of 24 times per day×30 days; (2) pulse: labeled array every minute during exercise/rest; (3) body temperature: scalar value once per day×30 days; (4) activity labels: category array for exercise, rest, meals, etc.; (5) environmental data: time-series tensor of room temperature, humidity, atmospheric pressure, etc. Examples of AI output include: (1) abnormality score for each category (e.g., blood pressure abnormality 0.8, pulse abnormality 0.2, body temperature abnormality 0.1); (2) event labels (e.g., hypertension, tachycardia, hypothermia, overactivity, etc.); (3) category-specific recommended actions (e.g., salt restriction for blood pressure abnormality, rest recommendation for pulse abnormality, hydration for body temperature abnormality, etc.). Based on the category-specific abnormality scores and event labels output by the AI, the determination unit provides optimized information for each category to subsequent advice generation units and emergency alert transmission units. By flexibly switching determination methods according to data diversity and complexity, unlike conventional uniform determination methods, technical effects such as improved determination accuracy, reduced false positives, and personalized health evaluation for each user are achieved. Application fields include lifestyle disease management, performance analysis for athletes, multifaceted health monitoring in home care, and corporate health management support. The technical effect is that the introduction of category-optimized determination by AI enables simultaneous realization of high-precision determination for multiple data types and improvement of user experience.

The determination unit is capable of estimating the user's emotion and adjusting the display method of determination results based on the estimated emotion of the user. For example, the determination unit uses AI to estimate the user's emotion and adjusts the display method of determination results according to the estimated emotion. Specific methods and criteria for emotion estimation may include facial recognition, voice analysis, self-reporting, and the like, but are not limited thereto. For instance, when the user is nervous, the determination unit provides a simple and highly visible display method. When the user is relaxed, the determination unit can provide a display method including detailed information. Furthermore, when the user is in a hurry, the determination unit can provide a display method focusing on key points. By adjusting the display method of determination results according to the user's emotion, more appropriate display methods can be provided. Emotion estimation is realized, for example, by using an emotion engine or a generative AI as an emotion estimation function. The generative AI may be a text generation AI (e.g., LLM) or a multimodal generative AI, but is not limited thereto. Some or all of the above-described processing in the determination unit may be performed using AI or without using AI. For example, the determination unit inputs the user's emotion data to AI, and the AI adjusts the display method of determination results. Specifically, the determination unit inputs multimodal emotion data such as the user's facial image (224×224×3), voice waveform (16000×1), and self-reported text (natural language sentence) to an emotion estimation AI (CNN+RNN+large language model), which outputs emotion labels (e.g., nervousness, relaxation, hurry, etc.) and emotion intensity scores (0.0-1.0). Examples of AI input include: (1) “Facial: frown, voice: low tone, text: feeling nervous”→nervousness; (2) “Facial: smile, voice: normal, text: feeling calm”→relaxation; (3) “Facial: serious, voice: fast, text: in a hurry”→hurry. Examples of AI output include: (1) emotion label; (2) emotion intensity score; (3) recommended display mode (e.g., simple display for nervousness, detailed display for relaxation, key point display for hurry, etc.). Based on the recommended display mode output by the AI, the user interface control module automatically adjusts the display content, layout, color scheme, and amount of information. For example, in a nervous state, graphs and numbers are minimized and only important abnormalities are highlighted; in a relaxed state, detailed time-series graphs and statistical information are additionally displayed; and in a hurry, only key points are displayed in large font. These outputs realize information presentation that responds to the user's psychological state and usage situation, and unlike conventional uniform UIs, provide technical effects such as preventing increased stress due to information overload and missing important information, and improving user experience. Application fields include stress management for chronic disease patients, on-site monitoring for athletes, UI optimization for elderly home care, and corporate health management dashboards. The technical effect is that the combination of emotion estimation and dynamic UI control by AI enables simultaneous realization of personalized information presentation and improved user satisfaction.

The determination unit is capable of determining the priority of determination based on the timing of health data collection at the time of determination. For example, the determination unit uses AI to determine the priority of determination according to the timing of health data collection at the time of determination. Specific methods and criteria for determining the priority of determination may include the timing and importance of health data collection, among others, but are not limited thereto. For instance, the determination unit prioritizes analysis of morning data to predict the user's health status for the day. The determination unit can also prioritize analysis of post-exercise data to evaluate exercise effects. Furthermore, the determination unit can prioritize analysis of nighttime data to evaluate sleep quality. By determining the priority of determination based on the timing of health data collection, more appropriate determinations can be made. Some or all of the above-described processing in the determination unit may be performed using AI or without using AI. For example, the determination unit inputs the timing of health data collection to AI, and the AI determines the priority of determination. Specifically, the determination unit obtains input tensors such as timestamps attached to each data (e.g., 2024-06-01 07:00:00), activity labels (post-exercise, before bedtime, nighttime), and user schedule information (calendar API integration), and inputs them to a time-series priority estimation AI (e.g., time-series classification model+rule-based priority determination module). Examples of AI input include: (1) “Blood pressure: 7 a.m., activity: immediately after waking”; (2) “Pulse: 10 minutes after exercise, activity: running”; (3) “Body temperature: 11 p.m., activity: before bedtime”. Examples of AI output include: (1) determination priority score (0.0-1.0); (2) recommended determination order list (e.g., morning data→post-exercise data→nighttime data); (3) determination purpose label (e.g., daytime prediction, exercise effect evaluation, sleep evaluation, etc.). Based on the priority score and recommended order list output by the AI, the determination scheduler automatically optimizes the data analysis order. For example, morning blood pressure data is analyzed with the highest priority as it is directly related to daily health risk prediction; post-exercise data is immediately input to the exercise effect evaluation module; and nighttime data is preferentially input to the sleep evaluation AI. These outputs realize determination that responds to the user's daily rhythm and health risks, and unlike conventional uniform batch analysis methods, provide technical effects such as preventing missing important events and improving real-time performance. Application fields include early sign detection of lifestyle diseases, performance management for athletes, nighttime monitoring for elderly at home, and corporate health management monitoring. The technical effect is that the introduction of time-series priority control by AI enables simultaneous realization of improved data utility and efficient use of determination resources.

The determination unit is capable of adjusting the order of determination based on the relevance of health data at the time of determination. For example, the determination unit uses AI to adjust the order of determination according to the relevance of health data at the time of determination. Specific methods and criteria for adjusting the order of determination may include the relevance and importance of health data, among others, but are not limited thereto. For instance, the determination unit associates and analyzes blood pressure and pulse data to evaluate cardiovascular health status. The determination unit can also associate and analyze body temperature and body weight data to evaluate metabolic health status. Furthermore, the determination unit can associate and analyze step count and body weight data to evaluate exercise effects. By adjusting the order of determination based on the relevance of health data, more accurate determinations can be made. Some or all of the above-described processing in the determination unit may be performed using AI or without using AI. For example, the determination unit inputs the relevance of health data to AI, and the AI adjusts the order of determination. Specifically, the determination unit obtains multidimensional time-series data such as blood pressure, pulse, body temperature, body weight, and step count as structured tensors (e.g., 5×24×1), and inputs them to a correlation analysis AI (e.g., multivariate regression model, graph neural network, causal inference AI, etc.) to calculate correlation coefficients, causal relationship scores, and importance indicators among the data. Examples of AI input include: (1) simultaneous time-series data of blood pressure and pulse; (2) one-month array of body temperature and body weight; (3) composite data set of step count, body weight, and exercise intensity. Examples of AI output include: (1) relevance score matrix (e.g., blood pressure-pulse 0.85, body temperature-body weight 0.65, etc.); (2) recommended determination order list (e.g., cardiovascular system→metabolic system→exercise system); (3) determination group labels (e.g., cardiovascular health, metabolic health, exercise effect, etc.). Based on the relevance scores and recommended order list output by the AI, the determination pipeline control module automatically optimizes the data analysis order and grouping. For example, when the correlation between blood pressure and pulse is high, cardiovascular health determination is performed with the highest priority; when the relevance between body temperature and body weight is strong, metabolic health determination is performed together; and when the relevance between step count and body weight is high, exercise effect determination is emphasized. These outputs contribute to comprehensive health evaluation and improved abnormality detection accuracy for multiple data, and unlike conventional sequential determination methods for single data, provide the technical effect of high-precision determination that considers interactions among data. Application fields include cardiovascular disease risk assessment, metabolic abnormality detection, performance analysis for athletes, and multifaceted health monitoring in home care. The technical effect is that the introduction of correlation analysis and order optimization by AI enables simultaneous realization of high-precision determination for multiple data types and improvement of user experience.

The linkage unit is capable of estimating the user's emotion and adjusting the linkage method based on the estimated emotion of the user. For example, the linkage unit uses AI to estimate the user's emotion and adjusts the linkage method according to the estimated emotion. Specific methods and criteria for emotion estimation may include facial recognition, voice analysis, self-reporting, and the like, but are not limited thereto. For instance, when the user is feeling stressed, the linkage unit shares information useful for stress reduction. When the user is relaxed, the linkage unit can share information useful for health maintenance. Furthermore, when the user is exercising, the linkage unit can share information to enhance exercise effects. By adjusting the linkage method according to the user's emotion, more appropriate linkage can be performed. Emotion estimation is realized, for example, by using an emotion engine or a generative AI as an emotion estimation function. The generative AI may be a text generation AI (e.g., LLM) or a multimodal generative AI, but is not limited thereto. Some or all of the above-described processing in the linkage unit may be performed using AI or without using AI. For example, the linkage unit inputs the user's emotion data to AI, and the AI adjusts the linkage method. Specifically, the linkage unit inputs multimodal emotion data such as the user's facial image (224×224×3), voice waveform (16000×1), and self-reported text (natural language sentence) to an emotion estimation AI (CNN+RNN+large language model), which outputs emotion labels (e.g., stress, relaxation, exercising, etc.) and emotion intensity scores (0.0-1.0). Examples of AI input include: (1) “Facial: frown, voice: low tone, text: high stress”→stress; (2) “Facial: smile, voice: normal, text: feeling calm”→relaxation; (3) “Facial: serious, voice: slightly high, text: exercising”→exercising. Examples of AI output include: (1) emotion label; (2) emotion intensity score; (3) recommended linkage method (e.g., share stress reduction information during stress, share health maintenance information during relaxation, share exercise effect information during exercise, etc.). Based on the recommended linkage method output by the AI, the linkage channel generation module automatically adjusts the information sharing content and notification method. For example, in a stress state, information useful for stress reduction is shared via group chat; in a relaxed state, lifestyle information for health maintenance is distributed; and during exercise, advice and encouragement messages to enhance exercise effects are sent. These outputs contribute to real-time mutual assistance and improved information sharing efficiency among users, and unlike conventional uniform information sharing methods, provide the technical effect of personalized linkage optimization that responds to the user's psychological state and activity situation. Application fields include peer support for chronic disease patients, regional monitoring networks, group guidance for corporate health management, and condition sharing for sports teams. The technical effect is that the combination of emotion estimation and dynamic linkage method control by AI enables simultaneous realization of autonomous support network formation among users and improved information sharing efficiency.

The linkage unit is capable of referring to past linkage history at the time of linkage to select an appropriate linkage method. For example, the linkage unit uses AI to refer to past linkage history at the time of linkage and select the optimal linkage method. Specific methods for referring to and utilizing linkage history may include past linkage content and results, among others, but are not limited thereto. For instance, the linkage unit proposes the optimal linkage method based on the user's previously used linkage methods. The linkage unit can also select effective linkage methods from the user's past linkage history. Furthermore, the linkage unit can analyze the user's past linkage history and propose the most efficient linkage method. By referring to past linkage history, the optimal linkage method can be selected. Some or all of the above-described processing in the linkage unit may be performed using AI or without using AI. For example, the linkage unit inputs past linkage history to AI, and the AI selects the optimal linkage method. Specifically, the linkage unit accumulates time-series recorded linkage history data for each user (e.g., linkage date and time, type of linkage means, attributes of linkage partners, linkage content text, user reactions and changes in health indicators after linkage, etc.) in a structured database on the server. The linkage unit inputs these history data to a time-series embedding model, graph neural network, or clustering AI to extract multidimensional features such as past linkage patterns and their effects (e.g., degree of stress reduction, improvement in health indicators, user satisfaction score, etc.). Examples of AI input include: (1) “2024-06-01 10:00:00, group chat, stress reduction information sharing, stress score decrease”; (2) “2024-06-02 20:00:00, individual message, exercise encouragement, increase in step count”; (3) “2024-06-03 08:00:00, health column distribution, health maintenance information, no user reaction”. Examples of AI output include: (1) recommended linkage method (e.g., group chat, individual message, information distribution, etc.); (2) recommended linkage timing (e.g., morning, night, after exercise, etc.); (3) predicted linkage effect score (0.0-1.0). Based on the recommended linkage method and timing output by the AI, the linkage channel control module automatically selects the optimal linkage means, content, and notification timing, and shares information with users or groups. These outputs realize personalized linkage optimization based on past history, and unlike conventional uniform linkage methods, provide the technical effect of maximizing user response and effect. History reference-type linkage optimization by AI is fundamentally different from conventional techniques in that it analyzes hundreds to thousands of history data in high-dimensional space to extract nonlinear causal relationships and hidden patterns, rather than relying on human memory or empirical rules. Application fields include peer support history optimization for chronic disease patients, group guidance history analysis for corporate health management, utilization of linkage history in regional monitoring networks, and condition sharing history optimization for sports teams. The technical effect is that the introduction of history-based linkage optimization by AI enables simultaneous realization of maximized linkage effect, improved user satisfaction, increased information sharing efficiency, and reduced linkage failure rate.

The linkage unit is capable of customizing the linkage means based on the user's current activity status at the time of linkage. For example, the linkage unit uses AI to grasp the user's current activity status at the time of linkage and customize the linkage means. Specific methods and criteria for customizing linkage means may include the means and content of linkage, among others, but are not limited thereto. For instance, when the user is exercising, the linkage unit shares information to enhance exercise effects. When the user is resting, the linkage unit can share information useful for relaxation. Furthermore, when the user is eating, the linkage unit can share information about healthy eating. By customizing the linkage means based on the user's current activity status, more appropriate linkage can be performed. Some or all of the above-described processing in the linkage unit may be performed using AI or without using AI. For example, the linkage unit inputs the user's current activity status to AI, and the AI customizes the linkage means. Specifically, the linkage unit obtains various activity context data in real time, such as activity labels (e.g., exercising, resting, eating, working, etc.) obtained from the collection unit or analysis unit, accelerometer data (3-axis acceleration: 100 Hz×3), GPS location information, schedule information via calendar API integration, voice commands, and touch operation logs. The linkage unit inputs these activity data to a convolutional neural network (CNN) or time-series classification AI to estimate the current activity state with high accuracy. Examples of AI input include: (1) “Acceleration: large fluctuation, GPS: moving, calendar: exercise scheduled”→exercising; (2) “Acceleration: slight, heart rate: stable, calendar: lunch”→eating; (3) “Acceleration: almost zero, body temperature: stable, calendar: bedtime”→resting. Examples of AI output include: (1) activity label (exercising, resting, eating, etc.); (2) recommended linkage means (e.g., share exercise effect information during exercise, distribute relaxation information during rest, notify healthy eating information during meals, etc.); (3) linkage priority score (0.0-1.0). Based on the activity label and recommended linkage means output by the AI, the linkage channel control module automatically optimizes the linkage content, notification means, and timing. For example, during exercise, encouragement messages and exercise effect explanations are shared in real time via group chat; during rest, relaxation audio guides and stretching videos are distributed; and during meals, healthy food suggestions and meal record sharing are performed. These outputs realize linkage optimization that responds to changes in user activity, and unlike conventional uniform linkage methods, provide technical effects such as situation-adaptive information sharing and enhanced support. Activity status recognition and linkage means customization by AI, unlike manual situation judgment and notification selection by humans, analyze multiple sensor data and time-series patterns in high-dimensional space to realize automatic optimization by rule-based processing and neural networks. Application fields include performance linkage at sports sites, lifestyle rhythm linkage in home care, work safety monitoring linkage in companies, and situation-adaptive support for lifestyle disease management. The technical effect is that the combination of activity recognition and linkage means control by AI enables situation-adaptive linkage optimization and improved information sharing efficiency for each user.

The linkage unit is capable of estimating the user's emotion and determining the priority of linkage based on the estimated emotion of the user. For example, the linkage unit uses AI to estimate the user's emotion and determines the priority of linkage according to the estimated emotion. Specific methods and criteria for emotion estimation may include facial recognition, voice analysis, self-reporting, and the like, but are not limited thereto. For instance, when the user is feeling stressed, the linkage unit prioritizes sharing information useful for stress reduction. When the user is relaxed, the linkage unit can prioritize sharing information useful for health maintenance. Furthermore, when the user is exercising, the linkage unit can prioritize sharing information to enhance exercise effects. By determining the priority of linkage according to the user's emotion, more appropriate linkage can be performed. Emotion estimation is realized, for example, by using an emotion engine or a generative AI as an emotion estimation function. The generative AI may be a text generation AI (e.g., LLM) or a multimodal generative AI, but is not limited thereto. Some or all of the above-described processing in the linkage unit may be performed using AI or without using AI. For example, the linkage unit inputs the user's emotion data to AI, and the AI determines the priority of linkage. Specifically, the linkage unit inputs multimodal emotion data such as the user's facial image (224×224×3), voice waveform (16000×1), and self-reported text (natural language sentence) to an emotion estimation AI (CNN+RNN+large language model), which outputs emotion labels (e.g., stress, relaxation, exercising, etc.) and emotion intensity scores (0.0-1.0). Examples of AI input include: (1) “Facial: frown, voice: low tone, text: feeling irritated”→stress; (2) “Facial: smile, voice: normal, text: feeling calm”→relaxation; (3) “Facial: serious, voice: slightly high, text: exercising”→exercising. Examples of AI output include: (1) emotion label; (2) emotion intensity score; (3) recommended linkage priority list (e.g., prioritize stress reduction information during stress, prioritize health maintenance information during relaxation, prioritize exercise effect information during exercise, etc.); (4) priority score (0.0-1.0). Based on the priority list output by the AI, the linkage channel control module automatically optimizes the linkage content, notification order, and timing. For example, in a stress state, stress reduction information is shared with the highest priority via group chat or individual notification; in a relaxed state, health maintenance information is distributed in detail; and during exercise, exercise effect explanations and encouragement messages are sent immediately. These outputs realize linkage priority control that responds to the user's psychological state and activity situation, and unlike conventional uniform linkage methods, provide technical effects such as preventing missing important information and improving user experience. Emotion estimation and dynamic linkage priority control by AI integrate diverse inputs such as facial expressions, voice, and text, and combine high-precision emotion recognition by neural networks with rule-based priority determination to realize personalized linkage optimization that is difficult with human intuitive judgment or simple rules. Application fields include stress management linkage for chronic disease patients, on-site support for athletes, linkage optimization for elderly home care, and group linkage for corporate health management. The technical effect is that the combination of emotion estimation and dynamic linkage priority control by AI enables simultaneous realization of personalized linkage support and improved information sharing efficiency.

The linkage unit is capable of selecting an appropriate linkage method by considering the user's geographic location information at the time of linkage. For example, the linkage unit uses AI to consider the user's geographic location information at the time of linkage and select the optimal linkage method. Specific methods and criteria for selecting the linkage method may include linkage methods based on geographic location information, among others, but are not limited thereto. For instance, when the user is in a high-altitude area, the linkage unit prioritizes the collection of oxygen saturation and heart rate data. When the user is in an urban area, the linkage unit can prioritize the collection of step count and stress level data. Furthermore, when the user is at home, the linkage unit can prioritize the collection of body temperature and body weight data. By considering the user's geographic location information, the optimal linkage method can be selected. Some or all of the above-described processing in the linkage unit may be performed using AI or without using AI. For example, the linkage unit inputs the user's geographic location information to AI, and the AI selects the optimal linkage method. Specifically, the linkage unit obtains latitude and longitude data from GPS sensors (e.g., 35.6895, 139.6917), altitude data, Wi-Fi/Bluetooth beacon information, and geographic features from map APIs (determination of urban area, high-altitude area, home, etc.) in real time. The linkage unit inputs these geographic information to a geographic feature extraction AI (e.g., geographic information embedding model+rule-based AI) to estimate the environmental characteristics of the user's current location. Examples of AI input include: (1) “Latitude/longitude: altitude 2000 m, surroundings: mountainous area”→high-altitude area; (2) “Latitude/longitude: city center, surroundings: office district”→urban area; (3) “Latitude/longitude: residential area, Wi-Fi: home SSID”→home. Examples of AI output include: (1) environment label (high-altitude area, urban area, home, etc.); (2) recommended linkage method (share oxygen saturation and heart rate data in high-altitude areas, share step count and stress level information in urban areas, share body temperature and body weight information at home, etc.); (3) linkage priority list. Based on the environment label and recommended linkage method output by the AI, the linkage channel control module automatically optimizes the linkage content, notification means, and timing. For example, in high-altitude areas, abnormality detection information for oxygen saturation and heart rate is shared immediately via group chat; in urban areas, changes in step count and stress level are notified in real time; and at home, health maintenance information for body temperature and body weight is distributed to family groups. These outputs realize linkage optimization that responds to the user's geographic environment, and unlike conventional uniform linkage methods, provide technical effects such as early detection of health risks due to environmental changes and improved information sharing efficiency. Geographic information analysis and linkage method control by AI, unlike human location recognition or empirical notification selection, analyze multiple geographic features in high-dimensional space to realize automatic optimization by neural networks and rule-based AI. Application fields include health management linkage for high-altitude climbers, stress monitoring linkage in urban areas, health management linkage for home care patients, and health risk monitoring linkage for travelers. The technical effect is that the combination of geographic information analysis and dynamic linkage method control by AI enables simultaneous realization of environment-adaptive linkage optimization and improved information sharing efficiency.

The linkage unit is capable of analyzing the user's social media activity at the time of linkage to propose linkage means. For example, the linkage unit uses AI to analyze the user's social media activity at the time of linkage and propose linkage means. Specific methods and criteria for proposing linkage means may include linkage means based on social media activity, among others, but are not limited thereto. For instance, when the user posts about feeling stressed on social media, the linkage unit prioritizes the collection of heart rate and blood pressure data. When the user posts about exercise, the linkage unit can prioritize the collection of step count and heart rate data. Furthermore, when the user posts about meals, the linkage unit can prioritize the collection of body weight and blood glucose data. By analyzing the user's social media activity, more appropriate linkage means can be proposed. Some or all of the above-described processing in the linkage unit may be performed using AI or without using AI. For example, the linkage unit inputs the user's social media activity to AI, and the AI proposes linkage means. Specifically, the linkage unit automatically obtains various metadata via API from multiple social media platforms used by the user, such as post text (natural language sentence), post time, posting frequency, presence of attached images or videos, and number of reactions (likes, comments, etc.). The linkage unit inputs these data to a pre-trained large language model or multimodal AI (e.g., text+image classification model) to perform emotion analysis of post content (e.g., category classification such as stress, exercise, meal, relaxation), activity trend scoring (e.g., number of posts per day, positive/negative ratio), and extraction of health-related events (e.g., key phrases such as “I'm tired today”, “I went running”, “I ate out”). Examples of AI input include: (1) post text “I'm tired from work today. Feeling stressed.”+post time+image (frown); (2) post text “Achieved 5 km morning run!”+GPS movement log; (3) post text “Dinner was grilled meat. Maybe I ate too much.”+meal image. Examples of AI output include: (1) emotion label (stress, exercise, meal, etc.); (2) health-related event tag (e.g., stress post, exercise post, meal post); (3) recommended linkage means list (e.g., share stress reduction information for stress posts, share exercise effect information for exercise posts, share meal management information for meal posts, etc.); (4) linkage priority score (0.0-1.0). Based on the event tag and recommended linkage means output by the AI, the linkage channel control module automatically optimizes the linkage content, notification means, and timing in real time. For example, when a stress post is detected, stress reduction information is shared via group chat; for exercise posts, exercise effect explanations and encouragement messages are distributed immediately; and for meal posts, meal management advice and meal record sharing are performed. By linking the user's online activity with linkage means, unlike conventional simple periodic linkage methods, personalized linkage optimization that responds to psychological and behavioral changes is realized. Social media analysis by AI combines natural language processing, image recognition, and time-series analysis for high-dimensional data processing, enabling real-time analysis of large numbers of posts and cross-platform linkage optimization that is difficult with manual processing. Technical effects include optimization of linkage means that respond to psychological and behavioral changes, improved early detection rate of abnormal signs, increased information sharing efficiency, and improved user experience. Application fields include mental health linkage, lifestyle disease management linkage, SNS-linked condition sharing for athletes, monitoring linkage for home care, and corporate health management support linkage.

The system according to the embodiment is not limited to the above examples and can be variously modified as follows, for example. Specifically, the system allows for diverse variations in both hardware and software, such as the configuration of various health management functions and AI modules, data flow, algorithm selection, user interface design, types of linkage channels, addition or modification of sensor devices, cloud linkage methods, data retention periods, and privacy control methods. The system can select and combine AI model architectures such as convolutional neural networks, recurrent neural networks, transformer models, graph neural networks, rule-based AI, and time-series clustering AI according to the application. In the data collection unit, the system can acquire data in real time or batch from various data sources such as wearable devices, smartphones, home IoT devices, and medical facility linkage APIs, and flexibly handle data formats such as time-series tensors, scalar values, category labels, image data, voice waveforms, and natural language text. The system can optimize data flow among modules such as the analysis unit, determination unit, provision unit, and linkage unit using methods such as message queues, event-driven systems, batch processing, and stream processing. For each function such as health status estimation, abnormality detection, advice generation, and linkage optimization by AI, the system utilizes multidimensional features such as user attributes, usage status, data category, emotional state, geographic location, activity history, and linkage history as input, and generates outputs such as abnormality scores, recommended actions, advice sentences, linkage means, priority lists, and display modes. These outputs are linked to subsequent processing such as user interface control, notification timing optimization, group linkage, medical facility linkage, data storage, and allocation of analysis resources. The technical effect is that the flexible changeability of system configuration and AI algorithms enables personalized health support that responds to user needs, environmental changes, and data diversity, as well as simultaneous realization of computational resource efficiency, scalability, maintainability, maximized abnormality detection accuracy, and information sharing efficiency. Application fields include lifestyle disease management, performance analysis for athletes, multifaceted health monitoring in home care, corporate health management support, regional monitoring networks, health risk monitoring for travelers, and can be applied to a wide range of use cases for individuals, groups, medical institutions, companies, and communities.

The health management system is capable of analyzing the user's sleep patterns and providing advice to improve sleep quality. For example, the analysis unit collects the user's sleep data and evaluates the depth and cycles of sleep. The provision unit generates specific advice to improve sleep quality and provides it to the user. Such advice may include relaxation methods before bedtime and suggestions for an appropriate bedroom environment. In addition, the determination unit can evaluate the impact of sleep deprivation on health and, if necessary, issue help alerts to medical facilities. Thus, the user's sleep quality can be improved and overall health status can be enhanced. Specifically, the health management system collects multimodal data in real time from wearable devices worn by the user or smartphone apps, such as accelerometer, heart rate sensor, skin temperature sensor, and ambient light sensor, and transmits these as time-series tensors (e.g., every minute×8 hours×multiple sensors=480×N dimensions) to the analysis unit. The analysis unit inputs these data to a sleep stage estimation AI (e.g., hybrid model of convolutional neural network and recurrent neural network), which outputs sleep stage labels for each time point (e.g., awake, light, deep, REM sleep), as well as sleep cycle patterns (e.g., repeated 90-minute cycles, deep sleep ratio, number of awakenings). Examples of AI input include: (1) accelerometer: slight fluctuation, heart rate: 50 bpm, skin temperature: 36.2° C.→deep sleep; (2) accelerometer: large fluctuation, heart rate: 70 bpm→awake; (3) ambient light: 0 lx→sleeping. Examples of AI output include: (1) time-series label array of sleep stages; (2) sleep indicators such as total sleep time, deep sleep ratio, number of awakenings; (3) sleep quality score (0.0-1.0). Based on the sleep indicators and quality score output by the AI, the provision unit uses a natural language generation AI or rule-based generation engine to generate specific advice sentences such as “Avoid using your smartphone before bed”, “Keep the room temperature around 20° C.”, “Do stretching before going to bed”, and immediately notifies the user terminal. The determination unit analyzes one month of past sleep data using an autoregressive RNN or time-series clustering AI, and when chronic sleep deprivation or abnormal patterns (e.g., decrease in deep sleep ratio, increase in number of awakenings) are detected, inputs abnormality scores and event labels to a threshold determination module, and, if necessary, issues help alerts to medical facilities via a medical facility linkage API. These outputs contribute to improving the user's sleep habits, reducing health risks, and strengthening cooperation with medical institutions, and, unlike conventional simple sleep time recording methods, provide technical effects such as improved sleep quality, health status, and early abnormality detection through multidimensional data analysis and personalized advice generation by AI. Application fields include early sign detection of insomnia and sleep apnea syndrome, condition management for athletes, nighttime monitoring for elderly at home, and corporate health management monitoring.

The health management system is capable of collecting a user's dietary data and evaluating nutritional balance. For example, the collection unit records the details of meals consumed by the user, and the analysis unit evaluates the nutritional balance based on this data. The provision unit generates specific meal suggestions to improve nutritional balance and provides them to the user. These may include suggestions for ingredients to increase vitamin or mineral intake, or advice on calorie restriction. Additionally, the determination unit evaluates the impact of nutritional imbalance on health and, if necessary, can issue help alerts to medical facilities. This enables improvement of the user's nutritional balance and enhancement of health status. Specifically, in this health management system, when the user records meal details using a smartphone application or wearable device, the collection unit acquires meal images (224×224×3 RGB images), meal content text (natural language sentences), intake time, quantity information, etc., and transmits these as multimodal input tensors to the analysis unit. The analysis unit automatically determines ingredient and dish categories using image recognition AI (convolutional neural network), extracts ingredients, cooking methods, and quantities from text using natural language processing AI (large language model), and calculates nutrient amounts such as calories, protein, fat, carbohydrates, vitamins, and minerals using nutrient estimation AI (rule-based+machine learning model). Examples of AI input include: (1) Image: salad+Text: “lettuce, tomato, chicken breast, olive oil”→high in vitamin C and protein; (2) Image: hamburger+Text: “with fries and cola”→high in fat and sugar; (3) Image: grilled fish set meal+Text: “rice, miso soup, spinach”→well-balanced, etc. Examples of AI output include: (1) nutrient balance score (0.0-1.0); (2) list of nutrient deficiencies or excesses (e.g., vitamin A deficiency, excess fat); (3) recommended ingredients and cooking methods list. The provision unit automatically generates specific meal suggestions such as “Vitamin A is insufficient today. Add carrots or spinach,” or “Fat intake is high, so fish dishes are recommended for dinner,” using natural language generation AI, and notifies the user's device. The determination unit analyzes one month of meal history and health indicators (weight, blood pressure, blood glucose, etc.) using time-series clustering AI, and if trends of nutritional imbalance or increased health risk are detected, inputs abnormal scores and event labels to a threshold determination module, and, if necessary, issues help alerts via a medical facility linkage API. These outputs contribute to improvement of users' eating habits, reduction of health risks, and strengthening of cooperation with medical institutions. Unlike conventional manual input or simple calorie calculation methods, the integration of AI-based image, text, and time-series data analysis and personalized suggestion generation provides technical effects such as improved nutritional balance, enhanced health status, and early detection of abnormalities. Application fields include prevention of lifestyle-related diseases, nutrition management for athletes, meal monitoring for homebound elderly, and support for corporate health management.

The health management system is capable of collecting a user's exercise data and evaluating the effectiveness of exercise. For example, the collection unit records the user's amount and type of exercise, and the analysis unit evaluates the effectiveness of exercise based on this data. The provision unit generates specific exercise suggestions to maximize exercise effectiveness and provides them to the user. These may include adjustments to exercise frequency or intensity, or suggestions for combinations of different exercises. Additionally, the determination unit evaluates the impact of insufficient exercise on health and, if necessary, can issue help alerts to medical facilities. This enables improvement of the user's exercise habits and enhancement of health status. Specifically, in this health management system, various exercise-related data such as acceleration sensor, gyroscope, GPS, and heart rate sensor data are collected in real time from wearable devices or smartphones worn by the user, and transmitted as time-series tensors (e.g., every second×30 minutes×4 sensors=1800×4 dimensions) to the analysis unit. The analysis unit inputs these data into an exercise type classification AI (convolutional neural network+time-series classification model), and outputs exercise type labels (walking, running, cycling, strength training, etc.), exercise intensity (METs value), calories burned, exercise duration, etc. Examples of AI input include: (1) Acceleration: constant, GPS: 2 km traveled→walking; (2) Acceleration: high-frequency variation, heart rate: 150 bpm→running; (3) Acceleration: intermittent, gyroscope: rotation→strength training, etc. Examples of AI output include: (1) exercise type label; (2) exercise intensity score; (3) estimated calories burned; (4) recommended exercise frequency and intensity list. The provision unit automatically generates specific exercise suggestions such as “Aerobic exercise is insufficient this week. Add walking three times a week,” or “Increase strength training intensity by 10%,” using natural language generation AI, and notifies the user's device. The determination unit analyzes one month of exercise history and health indicators (weight, blood pressure, body fat percentage, etc.) using time-series clustering AI, and if trends of insufficient or excessive exercise or increased health risk are detected, inputs abnormal scores and event labels to a threshold determination module, and, if necessary, issues help alerts via a medical facility linkage API. These outputs contribute to improvement of users' exercise habits, reduction of health risks, and strengthening of cooperation with medical institutions. Unlike conventional manual input or simple step count recording methods, the integration of AI-based multidimensional sensor data analysis and personalized exercise suggestion generation provides technical effects such as maximized exercise effectiveness, enhanced health status, and early detection of abnormalities. Application fields include prevention of lifestyle-related diseases, training management for athletes, exercise monitoring for homebound elderly, and support for corporate health management.

The health management system is capable of analyzing a user's stress level and providing advice for stress reduction. For example, the collection unit collects data such as the user's heart rate and blood pressure, and the analysis unit evaluates the stress level based on this data. The provision unit generates specific advice for stress reduction and provides it to the user. These may include suggestions for relaxation techniques or stress management methods. Additionally, the determination unit evaluates the impact of stress on health and, if necessary, can issue help alerts to medical facilities. This enables management of the user's stress level and improvement of health status. Specifically, in this health management system, multimodal stress-related data such as heart rate, heart rate variability (HRV), blood pressure, skin conductance, respiration rate, facial images, and voice waveforms are collected in real time from wearable devices or smartphones worn by the user, and transmitted as time-series tensors or image/voice arrays to the analysis unit. The analysis unit inputs these data into a stress estimation AI (convolutional neural network+recurrent neural network+large language model), and outputs a stress level score (0.0-1.0), stress event labels (e.g., high stress, normal, relaxed), and estimated stress factors (e.g., work, lack of exercise, lack of sleep). Examples of AI input include: (1) Heart rate: 90 bpm, HRV: low→high stress; (2) Blood pressure: 140/90, facial image: frown→stress; (3) Voice: fast speech, high pitch→stress, etc. Examples of AI output include: (1) stress level score; (2) stress event label; (3) recommended relaxation action list (e.g., deep breathing, meditation, stretching). The provision unit automatically generates specific stress reduction advice such as “Take five deep breaths,” “Use a meditation app before bedtime,” or “Play a stretching video,” using natural language generation AI, and notifies the user's device. The determination unit analyzes one month of stress history and health indicators (blood pressure, sleep data, weight, etc.) using time-series clustering AI, and if chronic high stress or increased health risk trends are detected, inputs abnormal scores and event labels to a threshold determination module, and, if necessary, issues help alerts via a medical facility linkage API. These outputs contribute to users' stress management, reduction of health risks, and strengthening of cooperation with medical institutions. Unlike conventional simple heart rate recording methods, the integration of AI-based multidimensional data analysis and personalized stress reduction suggestion generation provides technical effects such as stress level management, improved health status, and early detection of abnormalities. Application fields include mental health care, workplace stress management, condition management for athletes, stress monitoring for homebound elderly, and support for corporate health management.

The health management system is capable of analyzing a user's water intake and providing advice for appropriate hydration. For example, the collection unit records the user's water intake, and the analysis unit evaluates the water balance based on this data. The provision unit generates specific advice for appropriate hydration and provides it to the user. These may include daily water intake goals or suggestions for hydration timing. Additionally, the determination unit evaluates the impact of insufficient hydration on health and, if necessary, can issue help alerts to medical facilities. This enables management of the user's water balance and improvement of health status. Specifically, in this health management system, when the user records water intake using a smartphone application or wearable device, the collection unit acquires data such as beverage type (e.g., water, tea, sports drinks), intake amount (in ml), intake time, and activity status (during exercise, at rest, etc.), and transmits these as time-series tensors or category labels to the analysis unit. The analysis unit inputs these data into a water balance evaluation AI (rule-based+time-series analysis model), and outputs total daily intake, recommended intake goals (automatically calculated based on weight, activity level, temperature, etc.), and recommended hydration timing list (e.g., before/after exercise, after bathing). Examples of AI input include: (1) Intake: 500 ml, time: 10:00, activity: before exercise→pre-exercise hydration; (2) Intake: 200 ml, time: 15:00, activity: at rest→snack time hydration; (3) Intake: 0 ml, time: 12:00-18:00→dehydration warning, etc. Examples of AI output include: (1) total daily intake; (2) recommended intake goal; (3) recommended hydration timing list; (4) dehydration warning label. The provision unit automatically generates specific hydration advice such as “Today's water intake has not reached the goal. Please hydrate again in the evening,” or “Drink 500 ml of water within 30 minutes after exercise,” using natural language generation AI, and notifies the user's device. The determination unit analyzes one month of water intake history and health indicators (weight, body temperature, blood pressure, etc.) using time-series clustering AI, and if chronic dehydration or increased health risk trends are detected, inputs abnormal scores and event labels to a threshold determination module, and, if necessary, issues help alerts via a medical facility linkage API. These outputs contribute to users' water balance management, reduction of health risks, and strengthening of cooperation with medical institutions. Unlike conventional manual input or simple goal notification methods, the integration of AI-based multidimensional data analysis and personalized hydration suggestion generation provides technical effects such as optimized water balance, improved health status, and early detection of abnormalities. Application fields include heatstroke prevention, hydration management for athletes, dehydration prevention for homebound elderly, and support for corporate health management.

The health management system is capable of estimating a user's emotion and adjusting the content of health advice based on the estimated emotion. For example, the provision unit prioritizes advice for stress reduction when the user is feeling stressed. When the user is relaxed, it can provide advice for maintaining health. Furthermore, when the user is exercising, it can provide advice to enhance exercise effectiveness. This enables the provision of appropriate health advice according to the user's emotion. Specifically, in this health management system, multimodal emotion data such as facial images (224×224×3), voice waveforms (16000×1), and self-reported text (natural language sentences) are input into an emotion estimation AI (convolutional neural network+recurrent neural network+large language model), which outputs emotion labels (e.g., stress, relaxation, exercising) and emotion intensity scores (0.0-1.0). Examples of AI input include: (1) “Facial expression: frown, voice: low tone, text: feeling irritated”→stress; (2) “Facial expression: smile, voice: normal, text: feeling calm”→relaxation; (3) “Facial expression: serious, voice: slightly high, text: exercising”→exercising, etc. Examples of AI output include: (1) emotion label; (2) emotion intensity score; (3) recommended advice category (e.g., stress reduction, health maintenance, exercise effectiveness). The provision unit automatically generates specific advice sentences such as “Take a deep breath,” “Your health status is good today. Please continue your health management,” or “Add stretching after exercise,” using natural language generation AI or rule-based generation engines, and instantly notifies the user's device. These outputs enable personalized health support that responds immediately to the user's psychological state and activity status. Unlike conventional uniform template notifications, the combination of AI-based emotion estimation and dynamic control of advice content provides technical effects such as improved user satisfaction, promotion of behavioral change, and improvement of health status. Application fields include stress management for chronic disease patients, on-site advice for athletes, health support for home care of the elderly, and corporate health management dashboards.

The health management system is capable of estimating a user's emotion and adjusting the health data analysis method based on the estimated emotion. For example, the analysis unit prioritizes analysis of stress-related data when the user is feeling stressed. When the user is relaxed, it can analyze data to evaluate overall health status. Furthermore, when the user is exercising, it can analyze data to evaluate exercise effectiveness. This enables the application of appropriate analysis methods according to the user's emotion. Specifically, in this health management system, multimodal emotion data such as facial images, voice waveforms, and self-reported text are input into an emotion estimation AI (convolutional neural network+recurrent neural network+large language model), which outputs emotion labels and emotion intensity scores. The analysis unit dynamically switches the target data and analysis algorithms according to the emotion label output by the AI. For example, for a stress label, stress-related data such as heart rate, blood pressure, and HRV are preferentially input into time-series analysis AI or anomaly detection AI; for a relaxation label, overall health indicators such as body temperature, weight, and sleep data are input into comprehensive evaluation AI; and for an exercising label, exercise-related data such as step count, calories burned, and exercise intensity are input into exercise effectiveness evaluation AI. Examples of AI input include: (1) Emotion label: stress→heart rate and blood pressure data; (2) Emotion label: relaxation→body temperature and weight data; (3) Emotion label: exercising→step count and calories burned data, etc. Examples of AI output include: (1) category-specific anomaly score; (2) overall health status score; (3) exercise effectiveness indicators. These outputs are linked to subsequent advice generation units and alert issuing units, enabling optimization of analysis in response to the user's psychological state and activity status. Unlike conventional uniform analysis methods, the combination of AI-based emotion estimation and dynamic control of analysis methods provides technical effects such as improved analysis accuracy, reduced false alarm rate, and enhanced user experience. Application fields include mental health care, performance analysis for athletes, multidimensional health monitoring for home care, and support for corporate health management.

The health management system is capable of estimating a user's emotion and adjusting the frequency of health data collection based on the estimated emotion. For example, the collection unit increases the data collection frequency to obtain more detailed health status when the user is feeling stressed. When the user is relaxed, it can lower the data collection frequency to reduce the user's burden. Furthermore, when the user is exercising, it can increase the data collection frequency to record exercise effectiveness in detail. This enables adjustment of data collection frequency according to the user's emotion. Specifically, in this health management system, multimodal emotion data such as facial images, voice waveforms, and self-reported text are input into an emotion estimation AI (convolutional neural network+recurrent neural network+large language model), which outputs emotion labels and emotion intensity scores. The collection unit dynamically controls the data collection frequency parameters for various sensor data (heart rate, blood pressure, body temperature, step count, acceleration, etc.) according to the emotion label output by the AI. For example, for a stress label, the collection frequency of stress-related data such as heart rate and blood pressure is increased to every minute; for a relaxation label, the collection frequency of data such as body temperature and weight is reduced to once per day; and for an exercising label, the collection frequency of exercise-related data such as acceleration and step count is increased to every 10 seconds. Examples of AI input include: (1) Emotion label: stress→heart rate collected every minute; (2) Emotion label: relaxation→body temperature collected once per day; (3) Emotion label: exercising→acceleration collected every 10 seconds, etc. Examples of AI output include: (1) recommended collection frequency list; (2) collection priority score; (3) data category to be collected. These outputs are linked to the sensor control module of the collection unit, enabling optimization of data collection in response to the user's psychological state and activity status. Unlike conventional fixed collection interval methods, the combination of AI-based emotion estimation and dynamic control of collection frequency provides technical effects such as improved data utility, reduced user burden, and optimized battery consumption. Application fields include stress management for chronic disease patients, on-site monitoring for athletes, health management for home care of the elderly, and corporate health management dashboards.

The health management system is capable of estimating a user's emotion and determining the priority of health data to be collected based on the estimated emotion. For example, the collection unit prioritizes the collection of heart rate and blood pressure data when the user is feeling stressed. When the user is relaxed, it can prioritize the collection of body temperature and weight data. Furthermore, when the user is exercising, it can prioritize the collection of step count and heart rate data. This enables determination of the priority of data to be collected according to the user's emotion. Specifically, in this health management system, multimodal emotion data such as facial images, voice waveforms, and self-reported text are input into an emotion estimation AI (convolutional neural network+recurrent neural network+large language model), which outputs emotion labels and emotion intensity scores. The collection unit dynamically generates a collection priority list for various sensor data (heart rate, blood pressure, body temperature, weight, step count, etc.) according to the emotion label output by the AI, and collects and transmits data to the server in order of highest priority. For example, for a stress label, heart rate and blood pressure data are collected with highest priority; for a relaxation label, body temperature and weight data are prioritized; and for an exercising label, step count and heart rate data are prioritized. Examples of AI input include: (1) Emotion label: stress→prioritize heart rate and blood pressure; (2) Emotion label: relaxation→prioritize body temperature and weight; (3) Emotion label: exercising→prioritize step count and heart rate, etc. Examples of AI output include: (1) recommended collection priority list; (2) priority score; (3) data category to be collected. These outputs are linked to the sensor control module of the collection unit, enabling optimization of data collection in response to the user's psychological state and activity status. Unlike conventional uniform collection methods, the combination of AI-based emotion estimation and dynamic control of collection priority provides technical effects such as prevention of missing important data, improved data utility, and optimized battery consumption. Application fields include stress management for chronic disease patients, on-site monitoring for athletes, health management for home care of the elderly, and corporate health management dashboards.

The health management system is capable of estimating a user's emotion and adjusting the expression method of health advice based on the estimated emotion. For example, the provision unit provides advice in gentle language when the user is feeling stressed. When the user is relaxed, it can provide detailed advice. Furthermore, when the user is in a hurry, it can provide concise and focused advice. This enables adjustment of the expression method of advice according to the user's emotion. Specifically, in this health management system, multimodal emotion data such as facial images (224×224×3), voice waveforms (16000×1), and self-reported text (natural language sentences) are input into an emotion estimation AI (convolutional neural network+recurrent neural network+large language model), which outputs emotion labels (e.g., stress, relaxation, in a hurry) and emotion intensity scores (0.0-1.0). The provision unit automatically adjusts the tone, style, amount of information, and emphasis points of advice sentences using natural language generation AI or rule-based generation engines based on the emotion label output by the AI. For example, in a stress state, a gentle tone such as “Take it easy and rest slowly” is selected; in a relaxation state, detailed explanations such as “Your health status is good today. Please continue to maintain a balanced diet and moderate exercise” are added; and when in a hurry, only the key points such as “Don't forget to hydrate” are presented concisely. These outputs are instantly displayed as application or notification messages on the user's device, enabling personalized information presentation that responds to the user's psychological state and usage situation. Unlike conventional uniform template notifications, the combination of AI-based emotion estimation and dynamic control of advice expression provides technical effects such as improved user satisfaction, prevention of information overload, and prevention of missing important information. Application fields include stress management for chronic disease patients, on-site advice for athletes, UI optimization for health support for home care of the elderly, and corporate health management dashboards.

Below is a brief explanation of the processing flow of Example of the Embodiment. Specifically, in this system, each module—collection unit, analysis unit, provision unit, determination unit, and linkage unit—works in coordination to seamlessly realize the acquisition, analysis, advice generation, anomaly determination, and information sharing of user health data. The system optimizes data flow between modules using message queues and event-driven methods, and achieves high-precision data analysis, determination, advice generation, and linkage optimization through AI models. At each step, the types of input data for AI (e.g., time-series tensors, images, audio, natural language text), AI model architectures (e.g., convolutional neural networks, recurrent neural networks, large language models), AI output formats (e.g., anomaly scores, recommended actions, advice sentences, linkage means), and subsequent processing (e.g., threshold determination, advice generation, notification timing optimization, group linkage) are clearly designed to simultaneously realize personalized health support and efficient information sharing for each user. As a result, unlike conventional simple data recording and notification methods, the combination of AI-based multidimensional data analysis, dynamic control, and linkage optimization provides technical effects such as early detection of health anomalies, promotion of behavioral change, and improvement of user experience. Application fields include management of lifestyle-related diseases, performance analysis for athletes, multidimensional health monitoring for home care, support for corporate health management, and regional monitoring networks.
Step 1: The collection unit collects data from wearable devices worn by the user. The collected data includes blood pressure, pulse, step count, weight, body temperature, and so on. The collection unit acquires data from the wearable device in real time and transmits it to the AI. Step 2: The analysis unit analyzes the data collected by the collection unit. The analysis unit uses AI to analyze the data and evaluate the user's health status. For example, if blood pressure is high or pulse is abnormally fast, the analysis unit provides health advice based on the data. Step 3: The provision unit provides advice based on the analysis result obtained by the analysis unit. The provision unit uses AI to generate specific advice content and provides it to the user. For example, suggestions for meals or recommendations for exercise are included. Step 4: The determination unit instantly determines changes in chronic conditions and changes in health status. The determination unit uses AI to detect changes in chronic conditions and health status and provides appropriate support. For example, if a diabetic patient shows a sudden change in blood glucose level, the determination unit detects the change and issues help alerts to medical facilities. Step 5: The linkage unit realizes a social media integration function. The linkage unit uses AI to enable information sharing among users, allowing them to support each other. For example, users with the same chronic condition can share information, and in emergencies, alerts can be sent to nearby users to request help. Specifically, in this system, in Step 1, the collection unit acquires time-series tensor data such as blood pressure (24 times per day×30 days), pulse (every minute×24 hours×30 days), step count (once per day×30 days), weight and body temperature (once per day×30 days) from wearable devices or smartphones in real time, performs data preprocessing (noise removal, missing value completion, normalization, etc.), and transmits it to the AI analysis platform. In Step 2, the analysis unit inputs the data into AI models such as convolutional neural networks and recurrent neural networks, and outputs anomaly scores (0.0-1.0), event labels (hypertension, tachycardia, etc.), and recommended actions (salt restriction, rest, etc.). In Step 3, the provision unit generates personalized advice sentences such as “Your blood pressure is high today. Please reduce salt intake” using natural language generation AI or rule-based generation engines based on the AI output, and instantly notifies the user's device. In Step 4, the determination unit analyzes past data and chronic condition history using autoregressive RNNs and time-series clustering AI, and if abnormal trends or sudden changes are detected, inputs anomaly scores and event labels to a threshold determination module, and, if necessary, issues help alerts via a medical facility linkage API. In Step 5, the linkage unit analyzes multidimensional data such as user emotion, activity, geographic location, linkage history, and social media activity using AI, automatically selects optimal linkage means, timing, and content for group chat, individual messages, and information distribution, and realizes real-time information sharing and mutual support among users. These processing flows, through the combination of AI-based multidimensional data analysis, dynamic control, and linkage optimization, provide technical effects such as early detection of health anomalies, promotion of behavioral change, improvement of user experience, and strengthening of cooperation with medical institutions. Application fields include management of lifestyle-related diseases, performance analysis for athletes, multidimensional health monitoring for home care, support for corporate health management, and regional monitoring networks.

The specific processing unit 290 sends the results of specific processing to the smart device 14. In the smart device 14, the control unit 46A causes the output device 40 to output the results of specific processing. The microphone 38B acquires voice indicating user input in response to the results of specific processing. The control unit 46A sends the voice data indicating user input acquired by the microphone 38B to the data processing device 12. In the data processing device 12, the specific processing unit 290 acquires the voice data.

The data generation model 58 is a so-called generative AI (Artificial Intelligence). An example of the data generation model 58 is a generative AI such as ChatGPT (registered trademark) (Internet search <URL: https://openai.com/blog/chatgpt>). The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

Moreover, the processing by the data processing system 10 described above is executed by the specific processing unit 290 of the data processing device 12 or the control unit 46A of the smart device 14, but it may be executed by both the specific processing unit 290 of the data processing device 12 and the control unit 46A of the smart device 14. Additionally, the specific processing unit 290 of the data processing device 12 acquires or collects necessary information for processing from the smart device 14 or external devices, and the smart device 14 acquires or collects necessary information for processing from the data processing device 12 or external devices.

Each of the plurality of elements including the aforementioned collection unit, analysis unit, provision unit, determination unit, and linkage unit is implemented by at least one of, for example, a smart device 14 and a data processing apparatus 12. For example, the collection unit collects the user's health data using a camera 42 or a microphone 38B of the smart device 14 and transmits it to the data processing apparatus 12 via a control unit 46A. The analysis unit is implemented, for example, by a specific processing unit 290 of the data processing apparatus 12, analyzes the collected data, and evaluates the user's health status. The provision unit is implemented, for example, by the specific processing unit 290 of the data processing apparatus 12, generates specific advice based on the analysis result, and provides it to the user via a display 40A or a speaker 40B of the smart device 14. The determination unit is implemented, for example, by the specific processing unit 290 of the data processing apparatus 12, instantly determines changes in chronic conditions and health status, and issues help alerts to medical facilities as necessary. The linkage unit is implemented, for example, by the control unit 46A of the smart device 14, and realizes information sharing and support among users. The correspondence between each unit and the apparatus or control unit is not limited to the examples described above and various modifications are possible.

### Second Embodiment

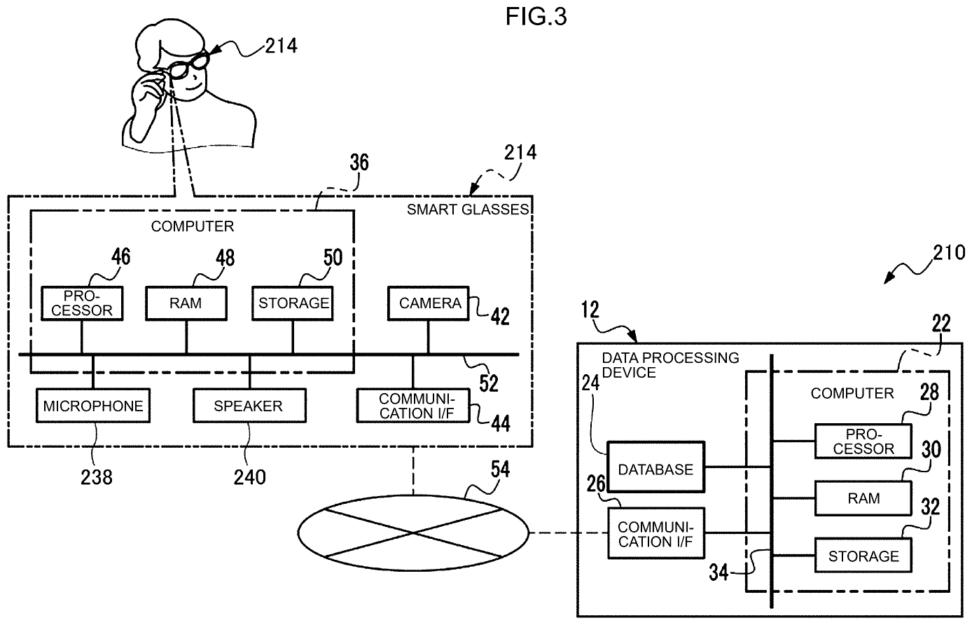

FIG. 3 shows an example configuration of a data processing system 210 according to the second embodiment.

As shown in FIG. 3, the data processing system 210 comprises a data processing device 12 and smart glasses 214. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The smart glasses 214 comprise a computer 36, a microphone 238, a speaker 240, a camera 42, and a communication I/F 44. The computer 36 comprises a processor 46, RAM 48, and storage 50. The processor 46, RAM 48, and storage 50 are connected to a bus 52. The microphone 238, speaker 240, and camera 42 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

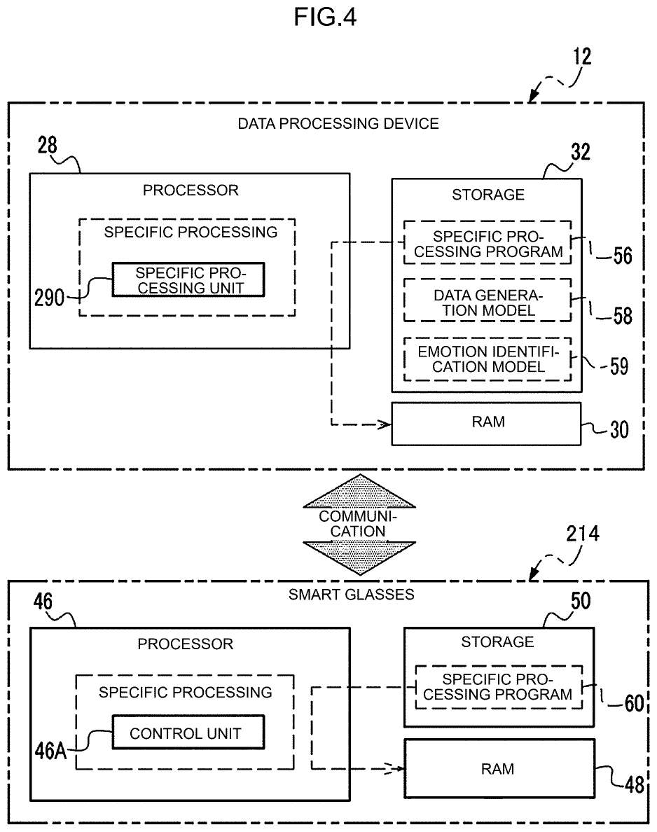

FIG. 4 shows an example of the main functions of the data processing device 12 and smart glasses 214. As shown in FIG. 4, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the smart glasses 214, specific processing is performed by the processor 46. The storage 50 stores a specific processing program 60. The processor 46 reads the specific processing program 60 from the storage 50 and executes it on the RAM 48. The specific processing is realized by the processor 46 operating as a control unit 46A according to the specific processing program 60 executed on the RAM 48. The smart glasses 214 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the smart glasses 214. In the smart glasses 214, the control unit 46A causes the speaker 240 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 210 according to the second embodiment performs the same processing as the data processing system 10 according to the first embodiment. The processing by the data processing system 210 is executed by the specific processing unit 290 of the data processing device 12 or the control unit 46A of the smart glasses 214, but it may be executed by both the specific processing unit 290 of the data processing device 12 and the control unit 46A of the smart glasses 214. Additionally, the specific processing unit 290 of the data processing device 12 acquires or collects necessary information for processing from the smart glasses 214 or external devices, and the smart glasses 214 acquires or collects necessary information for processing from the data processing device 12 or external devices.

Each of the plurality of elements including the aforementioned collection unit, analysis unit, provision unit, determination unit, and linkage unit is implemented by at least one of, for example, smart glasses 214 and a data processing apparatus 12. For example, the collection unit collects the user's health data using a camera 42 or a microphone 238 of the smart glasses 214 and transmits it to the data processing apparatus 12 via a control unit 46A. The analysis unit is implemented, for example, by a specific processing unit 290 of the data processing apparatus 12, analyzes the collected data, and evaluates the user's health status. The provision unit is implemented, for example, by the specific processing unit 290 of the data processing apparatus 12, generates specific advice based on the analysis result, and provides it to the user via a speaker 240 of the smart glasses 214. The determination unit is implemented, for example, by the specific processing unit 290 of the data processing apparatus 12, instantly determines changes in chronic conditions and health status, and issues help alerts to medical facilities as necessary. The linkage unit is implemented, for example, by the control unit 46A of the smart glasses 214, and realizes information sharing and support among users. The correspondence between each unit and the apparatus or control unit is not limited to the examples described above and various modifications are possible.

### Third Embodiment

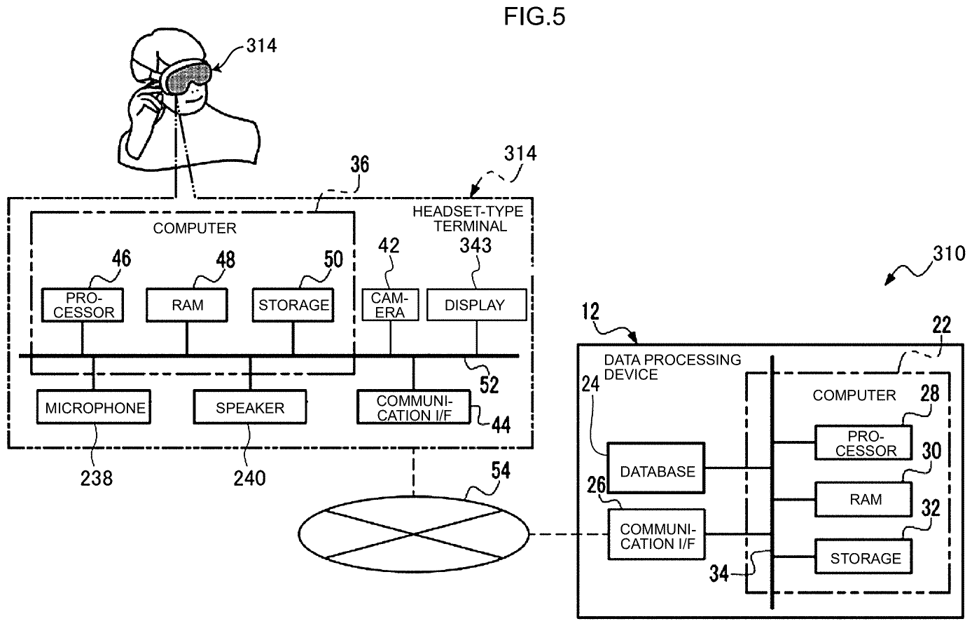

FIG. 5 shows an example configuration of a data processing system 310 according to the third embodiment.

As shown in FIG. 5, the data processing system 310 comprises a data processing device 12 and a headset-type terminal 314. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The headset-type terminal 314 comprises a computer 36, a microphone 238, a speaker 240, a camera 42, a communication I/F 44, and a display 343. The computer 36 comprises a processor 46, RAM 48, and storage 50. The processor 46, RAM 48, and storage 50 are connected to a bus 52. The microphone 238, speaker 240, camera 42, and display 343 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

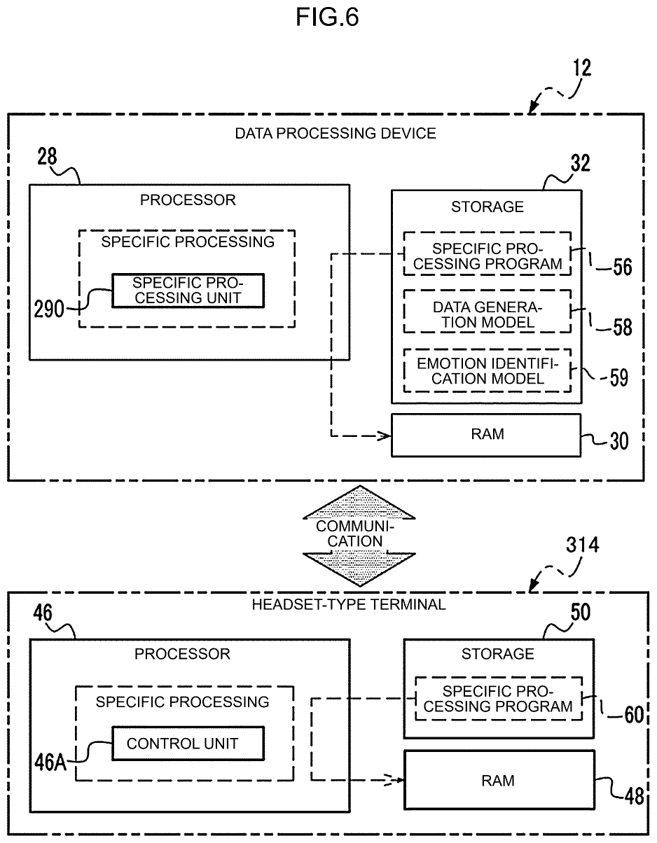

FIG. 6 shows an example of the main functions of the data processing device 12 and the headset-type terminal 314. As shown in FIG. 6, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the headset-type terminal 314, specific processing is performed by the processor 46. The storage 50 stores a specific program 60. The processor 46 reads the specific program 60 from the storage 50 and executes it on the RAM 48. The specific processing is realized by the processor 46 operating as a control unit 46A according to the specific program 60 executed on the RAM 48. The headset-type terminal 314 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the headset-type terminal 314. In the headset-type terminal 314, the control unit 46A causes the speaker 240 and the display 343 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 310 according to the third embodiment performs the same processing as the data processing system 10 according to the first embodiment. The processing by the data processing system 310 is executed by the specific processing unit 290 of the data processing device 12 or the control unit 46A of the headset-type terminal 314, but it may be executed by both the specific processing unit 290 of the data processing device 12 and the control unit 46A of the headset-type terminal 314. Additionally, the specific processing unit 290 of the data processing device 12 acquires or collects necessary information for processing from the headset-type terminal 314 or external devices, and the headset-type terminal 314 acquires or collects necessary information for processing from the data processing device 12 or external devices.

Each of the plurality of elements including the aforementioned collection unit, analysis unit, provision unit, determination unit, and linkage unit is implemented by at least one of, for example, a headset-type terminal 314 and a data processing apparatus 12. For example, the collection unit collects the user's health data using a camera 42 or a microphone 238 of the headset-type terminal 314 and transmits it to the data processing apparatus 12 via a control unit 46A. The analysis unit is implemented, for example, by a specific processing unit 290 of the data processing apparatus 12, analyzes the collected data, and evaluates the user's health status. The provision unit is implemented, for example, by the specific processing unit 290 of the data processing apparatus 12, generates specific advice based on the analysis result, and provides it to the user via a speaker 240 of the headset-type terminal 314. The determination unit is implemented, for example, by the specific processing unit 290 of the data processing apparatus 12, instantly determines changes in chronic conditions and health status, and issues help alerts to medical facilities as necessary. The linkage unit is implemented, for example, by the control unit 46A of the headset-type terminal 314, and realizes information sharing and support among users. The correspondence between each unit and the apparatus or control unit is not limited to the examples described above and various modifications are possible.

### Fourth Embodiment

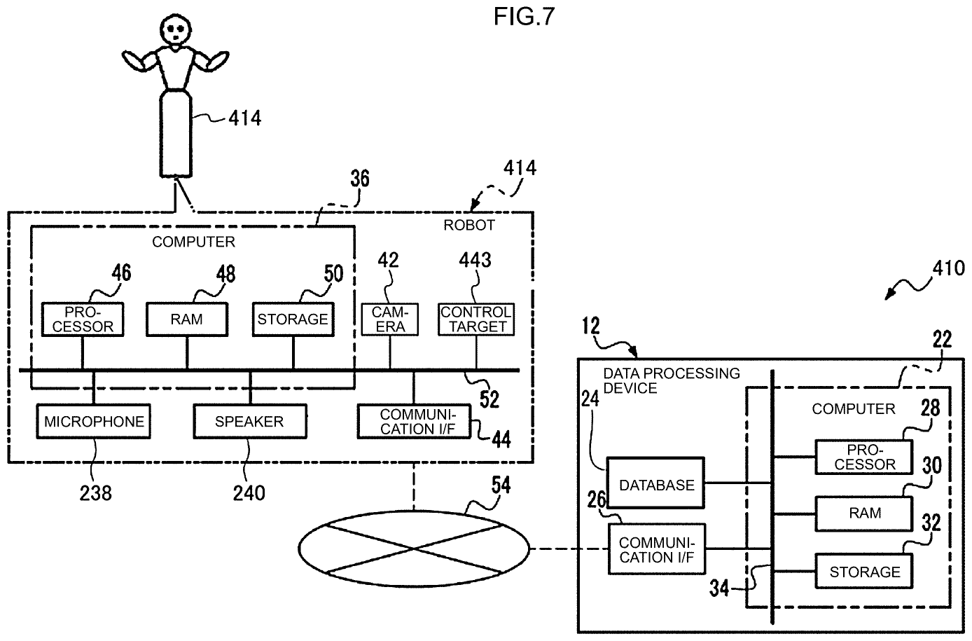

FIG. 7 shows an example configuration of a data processing system 410 according to the fourth embodiment.

As shown in FIG. 7, the data processing system 410 comprises a data processing device 12 and a robot 414. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The robot 414 comprises a computer 36, a microphone 238, a speaker 240, a camera 42, a communication I/F 44, and a control target 443. The computer 36 comprises a processor 46, RAM 48, and storage 50. The processor 46, RAM 48, and storage 50 are connected to a bus 52. The microphone 238, speaker 240, camera 42, and control target 443 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS image sensors or CCD image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

The control target 443 includes a display device, LEDs for the eyes, and motors for driving arms, hands, and feet, among others. The posture and gestures of the robot 414 are controlled by controlling the motors for the arms, hands, and feet, among others. Some emotions of the robot 414 can be expressed by controlling these motors. Additionally, the expression of the robot 414 can be expressed by controlling the lighting state of the LEDs for the eyes of the robot 414.

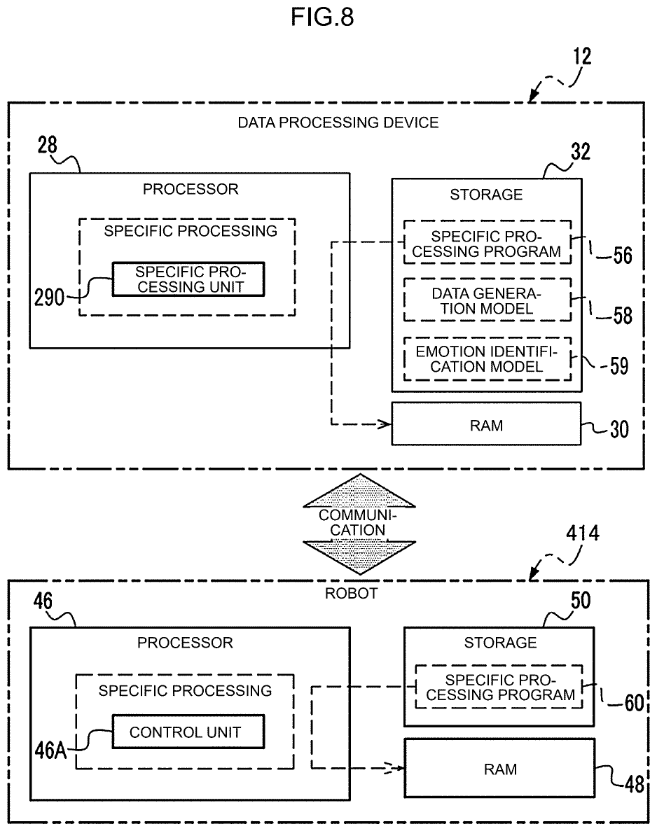

FIG. 8 shows an example of the main functions of the data processing device 12 and the robot 414. As shown in FIG. 8, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the robot 414, specific processing is performed by the processor 46. The storage 50 stores a specific program 60. The processor 46 reads the specific program 60 from the storage 50 and executes it on the RAM 48. The specific processing is realized by the processor 46 operating as a control unit 46A according to the specific program 60 executed on the RAM 48. The robot 414 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the robot 414. In the robot 414, the control unit 46A causes the speaker 240 and the control target 443 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 410 according to the fourth embodiment performs the same processing as the data processing system 10 according to the first embodiment. The processing by the data processing system 410 is executed by the specific processing unit 290 of the data processing device 12 or the control unit 46A of the robot 414, but it may be executed by both the specific processing unit 290 of the data processing device 12 and the control unit 46A of the robot 414. Additionally, the specific processing unit 290 of the data processing device 12 acquires or collects necessary information for processing from the robot 414 or external devices, and the robot 414 acquires or collects necessary information for processing from the data processing device 12 or external devices.

Each of the plurality of elements including the aforementioned collection unit, analysis unit, provision unit, determination unit, and linkage unit is implemented by at least one of, for example, a robot 414 and a data processing apparatus 12. For example, the collection unit collects the user's health data using a camera 42 or a microphone 238 of the robot 414 and transmits it to the data processing apparatus 12 via a control unit 46A. The analysis unit is implemented, for example, by a specific processing unit 290 of the data processing apparatus 12, analyzes the collected data, and evaluates the user's health status. The provision unit is implemented, for example, by the specific processing unit 290 of the data processing apparatus 12, generates specific advice based on the analysis result, and provides it to the user via a speaker 240 of the robot 414. The determination unit is implemented, for example, by the specific processing unit 290 of the data processing apparatus 12, instantly determines changes in chronic conditions and health status, and issues help alerts to medical facilities as necessary. The linkage unit is implemented, for example, by the control unit 46A of the robot 414, and realizes information sharing and support among users. The correspondence between each unit and the apparatus or control unit is not limited to the examples described above and various modifications are possible.

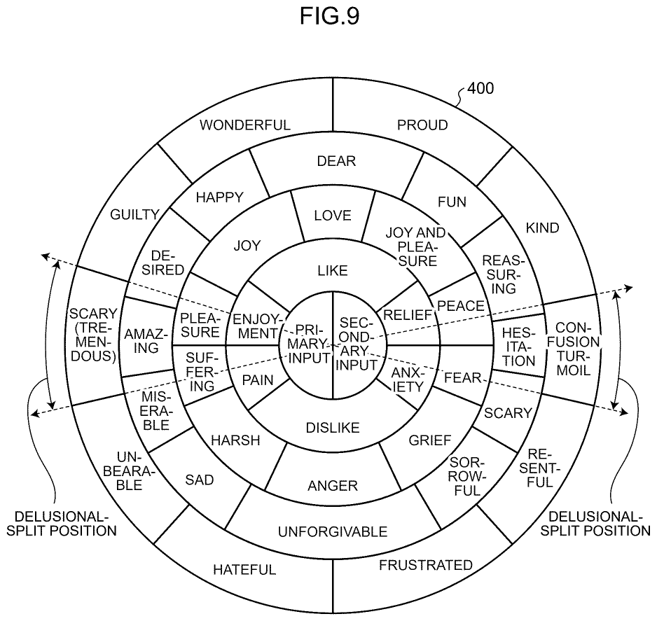

Note that the emotion identification model 59 as an emotion engine may determine the user's emotions according to a specific mapping. Specifically, the emotion identification model 59 may determine the user's emotions according to an emotion map, which is a specific mapping (see FIG. 9). Similarly, the emotion identification model 59 may determine the robot's emotions, and the specific processing unit 290 may perform specific processing using the robot's emotions.

FIG. 9 is a diagram showing an emotion map 400 where multiple emotions are mapped. In the emotion map 400, emotions are arranged concentrically radiating from the center. The closer to the center of the concentric circles, the more primitive the state of emotions is arranged. On the outer side of the concentric circles, emotions representing states and behaviors arising from mood are arranged. Emotions encompass concepts including emotional and mental states. On the left side of the concentric circles, emotions generally generated from reactions occurring in the brain are arranged. On the right side of the concentric circles, emotions generally induced by situational judgment are arranged. On the top and bottom of the concentric circles, emotions generated from reactions occurring in the brain and induced by situational judgment are arranged. Additionally, on the upper side of the concentric circles, “pleasant” emotions are arranged, and on the lower side, “unpleasant” emotions are arranged. In this way, in the emotion map 400, multiple emotions are mapped based on the structure from which emotions arise, and emotions that tend to occur simultaneously are mapped nearby.

These emotions are distributed in the 3 o'clock direction of the emotion map 400, and they usually move back and forth around reassurance and anxiety. In the right half of the emotion map 400, situational recognition takes precedence over internal sensations, giving a calm impression.

The inner side of the emotion map 400 represents the mind, and the outer side represents behavior, so the further out on the emotion map 400, the more visible (expressed in behavior) emotions become.

Here, human emotions are based on various balances like posture and blood sugar levels, and when these balances move away from the ideal, they indicate discomfort, and when they approach the ideal, they indicate comfort. In robots, cars, motorcycles, etc., emotions can be created based on various balances like posture and battery level, indicating discomfort when these balances move away from the ideal and comfort when they approach the ideal. The emotion map may be generated based on Dr. Mitsuyoshi's emotion map (Research on speech emotion recognition and brain physiological signal analysis systems related to emotions, Tokushima University, Doctoral dissertation: https://ci.nii.ac.jp/naid/500000375379). In the left half of the emotion map, emotions belonging to the domain called “reactions,” where sensations take precedence, are aligned. Additionally, in the right half of the emotion map, emotions belonging to the domain called “situations,” where situational recognition takes precedence, are aligned.

In the emotion map, two emotions that promote learning are defined. One is a negative emotion around “repentance” or “reflection” on the situation side. In other words, when a negative emotion arises in the robot, like “I never want to feel this way again” or “I don't want to be scolded again.” The other is an emotion around “desire” on the reaction side, which is positive. In other words, it is a positive feeling like “I want more” or “I want to know more.”

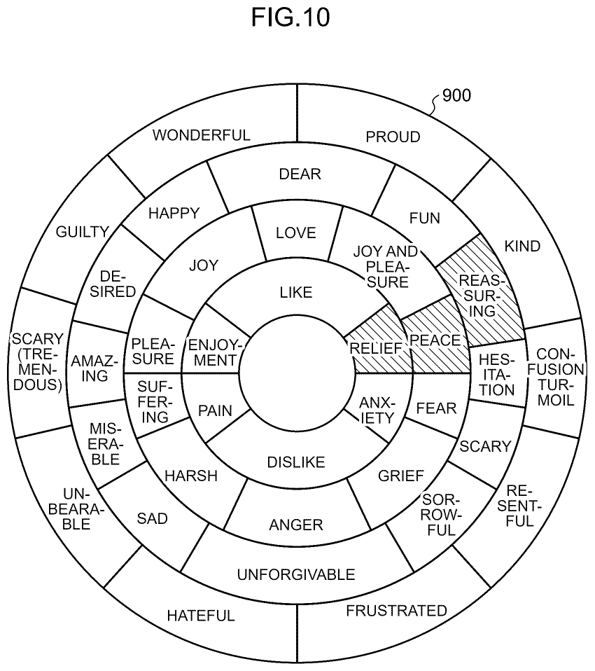

The emotion identification model 59 inputs user input into a pre-learned neural network, acquires emotion values indicating each emotion shown in the emotion map 400, and determines the user's emotions. This neural network is pre-learned based on multiple training data consisting of user input and combinations of emotion values indicating each emotion shown in the emotion map 400. Additionally, this neural network is learned so that emotions placed near each other in the emotion map 900 shown in FIG. 10 have similar values. FIG. 10 shows an example where multiple emotions like “reassured,” “calm,” and “confident” have similar emotion values.

In the above embodiments, an example form where specific processing is performed by a single computer 22 was described, but the technology disclosed herein is not limited to this, and distributed processing for specific processing by multiple computers including the computer 22 may be performed.

In the above embodiments, an example form where the specific processing program 56 is stored in the storage 32 was described, but the technology disclosed herein is not limited to this. For example, the specific processing program 56 may be stored in portable non-transitory storage media readable by a computer, such as a USB (Universal Serial Bus) memory. The specific processing program 56 stored in non-transitory storage media is installed in the computer 22 of the data processing device 12. The processor 28 executes specific processing according to the specific processing program 56.

Additionally, the specific processing program 56 may be stored in a storage device, such as a server connected to the data processing device 12 via the network 54, and downloaded and installed on the computer 22 in response to requests from the data processing device 12.

Furthermore, it is not necessary to store all of the specific processing program 56 in storage devices such as servers connected to the data processing device 12 via the network 54 or all in the storage 32, and a part of the specific processing program 56 may be stored.

Various processors, as shown next, can be used as hardware resources for executing specific processing. As processors, general-purpose processors that function as hardware resources for executing specific processing by executing software, i.e., programs, such as a CPU, can be mentioned. Additionally, as processors, dedicated electrical circuits with circuit configurations specially designed to execute specific processing, such as FPGA (Field-Programmable Gate Array), PLD (Programmable Logic Device), or ASIC (Application Specific Integrated Circuit), can be mentioned. Each processor has a built-in or connected memory, and each processor executes specific processing using the memory.

Hardware resources for executing specific processing may be composed of one of these various processors or a combination of two or more processors of the same or different types (e.g., a combination of multiple FPGAs or a combination of a CPU and FPGA). Additionally, hardware resources for executing specific processing may be a single processor.

As an example of composing with a single processor, firstly, there is a form where one or more CPUs and software are combined to constitute a single processor, which functions as hardware resources for executing specific processing. Secondly, there is a form using a processor, such as SoC (System-on-a-chip), that realizes the function of an entire system including multiple hardware resources for executing specific processing with a single IC chip. In this way, specific processing is realized using one or more of the various processors as hardware resources.

Furthermore, as a hardware structure of these various processors, more specifically, electrical circuits combined with circuit elements such as semiconductor elements can be used. Additionally, the specific processing described above is merely one example. Therefore, it goes without saying that unnecessary steps may be deleted, new steps may be added, or the order of processing may be changed within the scope not departing from the gist.

Additionally, in the examples described above, the explanation was divided into the first embodiment to the fourth embodiment, but parts or all of these embodiments may be combined. Additionally, the smart device 14, smart glasses 214, headset-type terminal 314, and robot 414 are examples, and each may be combined, or other devices may be used.

The descriptions and drawings shown above are detailed explanations of parts related to the technology disclosed herein and are merely examples of the technology disclosed herein. For example, the explanations regarding configurations, functions, actions, and effects above are explanations regarding examples of configurations, functions, actions, and effects of parts related to the technology disclosed herein. Therefore, it goes without saying that within the scope not departing from the gist of the technology disclosed herein, unnecessary parts may be deleted, new elements may be added, or replacements may be made to the descriptions and drawings shown above. Additionally, to avoid complexity and facilitate understanding of parts related to the technology disclosed herein, explanations concerning technical common knowledge and the like that do not require special explanation for enabling the implementation of the technology disclosed herein are omitted in the descriptions and drawings shown above.

All documents, patent applications, and technical standards described in this specification are incorporated by reference to the same extent as if each document, patent application, and technical standard were specifically and individually stated to be incorporated by reference in this specification.
(Supplementary Note 1)A system comprising: a collection unit configured to collect data; an analysis unit configured to analyze data collected by the collection unit; a provision unit configured to provide advice based on an analysis result obtained by the analysis unit; a determination unit configured to instantly determine changes in chronic conditions; a determination unit configured to instantly determine changes in health status; and a linkage unit configured to realize a social media integration function.
(Supplementary Note 2)The system according to Supplementary Note 1, wherein the collection unit is configured to transmit data in real time.
(Supplementary Note 3)The system according to Supplementary Note 1, wherein the analysis unit is configured to detect abnormal values.
(Supplementary Note 4)The system according to Supplementary Note 1, wherein the provision unit is configured to generate specific advice content.
(Supplementary Note 5)The system according to Supplementary Note 1, wherein the determination unit is configured to issue help alerts to medical facilities.
(Supplementary Note 6)The system according to Supplementary Note 1, wherein the linkage unit is configured to provide an information sharing function among users.

(Supplementary Note 7)The system according to Supplementary Note 1, wherein the collection unit is configured to estimate a user's emotion and adjust the data collection frequency based on the estimated emotion of the user.
(Supplementary Note 8)The system according to Supplementary Note 1, wherein the collection unit is configured to analyze a user's past health data and select an appropriate collection timing.
(Supplementary Note 9)The system according to Supplementary Note 1, wherein the collection unit is configured to change the type of data to be collected based on the user's current activity status at the time of data collection.
(Supplementary Note 10)The system according to Supplementary Note 1, wherein the collection unit is configured to estimate a user's emotion and determine the priority of data to be collected based on the estimated emotion of the user.
(Supplementary Note 11)The system according to Supplementary Note 1, wherein the collection unit is configured to preferentially collect highly relevant data by considering the user's geographic location information at the time of data collection.
(Supplementary Note 12)The system according to Supplementary Note 1, wherein the collection unit is configured to analyze the user's social media activity and collect related data at the time of data collection.
(Supplementary Note 13)The system according to Supplementary Note 1, wherein the analysis unit is configured to estimate a user's emotion and adjust the analysis algorithm based on the estimated emotion of the user.
(Supplementary Note 14)The system according to Supplementary Note 1, wherein the analysis unit is configured to refer to past health data during analysis to improve the accuracy of abnormal value detection.
(Supplementary Note 15)The system according to Supplementary Note 1, wherein the analysis unit is configured to apply different analysis methods for each data category during analysis.
(Supplementary Note 16)The system according to Supplementary Note 1, wherein the analysis unit is configured to estimate a user's emotion and adjust the display method of analysis results based on the estimated emotion of the user.
(Supplementary Note 17)The system according to Supplementary Note 1, wherein the analysis unit is configured to determine the priority of analysis based on the data collection timing during analysis.
(Supplementary Note 18)The system according to Supplementary Note 1, wherein the analysis unit is configured to adjust the order of analysis based on the relevance of the data during analysis.
(Supplementary Note 19)The system according to Supplementary Note 1, wherein the provision unit is configured to estimate a user's emotion and adjust the expression method of advice based on the estimated emotion of the user.
(Supplementary Note 20)The system according to Supplementary Note 1, wherein the provision unit is configured to adjust the level of detail of advice based on the importance of health data when providing advice.
(Supplementary Note 21)The system according to Supplementary Note 1, wherein the provision unit is configured to apply different advice algorithms according to the category of health data when providing advice.
(Supplementary Note 22)The system according to Supplementary Note 1, wherein the provision unit is configured to estimate a user's emotion and adjust the length of advice based on the estimated emotion of the user.
(Supplementary Note 23)The system according to Supplementary Note 1, wherein the provision unit is configured to determine the priority of advice based on the health data collection timing when providing advice.
(Supplementary Note 24)The system according to Supplementary Note 1, wherein the provision unit is configured to adjust the order of advice based on the relevance of health data when providing advice.
(Supplementary Note 25)The system according to Supplementary Note 1, wherein the determination unit is configured to estimate a user's emotion and adjust the determination criteria based on the estimated emotion of the user.
(Supplementary Note 26)The system according to Supplementary Note 1, wherein the determination unit is configured to refer to past health data during determination to improve the accuracy of determination.
(Supplementary Note 27)The system according to Supplementary Note 1, wherein the determination unit is configured to apply different determination methods for each health data category during determination.
(Supplementary Note 28)The system according to Supplementary Note 1, wherein the determination unit is configured to estimate a user's emotion and adjust the display method of determination results based on the estimated emotion of the user.
(Supplementary Note 29)The system according to Supplementary Note 1, wherein the determination unit is configured to determine the priority of determination based on the health data collection timing during determination.
(Supplementary Note 30)The system according to Supplementary Note 1, wherein the determination unit is configured to adjust the order of determination based on the relevance of health data during determination.
(Supplementary Note 31)The system according to Supplementary Note 1, wherein the linkage unit is configured to estimate a user's emotion and adjust the linkage method based on the estimated emotion of the user.
(Supplementary Note 32)The system according to Supplementary Note 1, wherein the linkage unit is configured to refer to past linkage history during linkage to select an appropriate linkage method.
(Supplementary Note 33)The system according to Supplementary Note 1, wherein the linkage unit is configured to customize the linkage means based on the user's current activity status during linkage.
(Supplementary Note 34)The system according to Supplementary Note 1, wherein the linkage unit is configured to estimate a user's emotion and determine the priority of linkage based on the estimated emotion of the user.
(Supplementary Note 35)The system according to Supplementary Note 1, wherein the linkage unit is configured to select an appropriate linkage method by considering the user's geographic location information during linkage.
(Supplementary Note 36)The system according to Supplementary Note 1, wherein the linkage unit is configured to analyze the user's social media activity and propose linkage means during linkage.

What is claimed is:

1. A system comprising:
circuitry configured to:
receive, from a client terminal via a communication interface and a packet-switched network, sensor data comprising at least one of time-series numerical data, voice data, or image data;
store the sensor data in a database as structured tensor data;
analyze the sensor data using a time-series analysis model comprising at least one of a convolutional neural network, a recurrent neural network, or a Transformer-based model to generate an anomaly score;
estimate an emotion of a user by applying an emotion identification model to the sensor data;
generate, using a data generation model obtained by deep learning on a neural network, inference data comprising at least one of a recommendation label, a probability score, or natural language text, based on the anomaly score and the estimated emotion;
transmit, when the anomaly score exceeds a threshold, an alert to an external system via the communication interface; and
transmit the inference data to the client terminal via the communication interface and the packet-switched network, the inference data causing the client terminal to present the inference data to the user.

2. The system according to claim 1, wherein the sensor data comprises health data obtained from a wearable device communicatively coupled to the client terminal, the health data comprising at least one of blood pressure, pulse, step count, body weight, or body temperature as time-series tensor data.

3. The system according to claim 1, wherein the circuitry is further configured to preprocess the sensor data by performing at least one of noise removal, missing value imputation, or normalization before analyzing the sensor data.

4. The system according to claim 1, wherein the time-series analysis model comprises an autoregressive recurrent neural network or a time-series clustering model, and wherein the circuitry is configured to extract features comprising at least one of periodicity, abnormal tendency, or peak occurrence time from the sensor data.

5. The system according to claim 1, wherein the circuitry is further configured to adjust a frequency of receiving the sensor data from the client terminal based on the estimated emotion, such that when the estimated emotion indicates stress, the circuitry increases the frequency, and when the estimated emotion indicates relaxation, the circuitry decreases the frequency.

6. The system according to claim 1, wherein the circuitry is further configured to analyze past sensor data stored in the database associated with the user to select an optimal data collection timing using at least one of a decision tree, a random forest, or a reinforcement learning model.

7. The system according to claim 1, wherein the circuitry is further configured to determine a type of sensor data to receive based on an activity status of the user, the activity status comprising at least one of exercising, resting, or eating, the activity status being estimated using an activity classification model applied to accelerometer data received from the client terminal.

8. The system according to claim 1, wherein the circuitry is further configured to determine a priority of sensor data to receive based on the estimated emotion, such that when the estimated emotion indicates stress, the circuitry prioritizes receiving sensor data associated with a stress-related attribute.

9. The system according to claim 1, wherein the circuitry is further configured to receive geographic location information from the client terminal and to adjust a type of sensor data to receive based on an environment label determined from the geographic location information, the environment label comprising at least one of high-altitude area, urban area, or home.

10. The system according to claim 1, wherein the circuitry is further configured to receive social media activity data from the client terminal, analyze the social media activity data using a natural language processing model to extract an emotion label, and adjust a type of sensor data to receive based on the extracted emotion label.

11. The system according to claim 1, wherein the circuitry is further configured to adjust an analysis algorithm based on the estimated emotion, such that when the estimated emotion indicates stress, the circuitry applies an algorithm that prioritizes analysis of stress-related data, and when the estimated emotion indicates relaxation, the circuitry applies an algorithm that evaluates overall status.

12. The system according to claim 1, wherein the circuitry is further configured to apply different analysis algorithms according to a category of the sensor data, such that for image data, the circuitry applies a convolutional neural network, for time-series numerical data, the circuitry applies a recurrent neural network, and for voice data, the circuitry applies a voice emotion recognition model.

13. The system according to claim 1, wherein the circuitry is further configured to adjust a display method of the inference data based on the estimated emotion, such that when the estimated emotion indicates stress, the circuitry generates the inference data in a simplified format, and when the estimated emotion indicates relaxation, the circuitry generates the inference data in a detailed format.

14. The system according to claim 1, wherein the circuitry is further configured to adjust a length of the inference data based on the estimated emotion, such that when the estimated emotion indicates urgency, the circuitry generates concise inference data, and when the estimated emotion indicates relaxation, the circuitry generates detailed inference data.

15. The system according to claim 1, wherein the circuitry is further configured to apply different inference data generation algorithms according to a category of the sensor data when generating the inference data.

16. The system according to claim 1, wherein the circuitry is further configured to determine a priority of generating the inference data based on a timestamp associated with the sensor data, such that sensor data having a more recent timestamp is processed with a higher priority.

17. The system according to claim 1, wherein the circuitry is further configured to cluster anonymized metadata of a plurality of users using a clustering algorithm to generate user groups with similar tendencies, and to transmit group notification data to client terminals associated with the user groups via the communication interface.

18. A system comprising:
a communication interface configured to communicate, via a packet-switched network conforming to at least one of a 5G, Wi-Fi, or Bluetooth communication standard, with a client terminal comprising a touch panel, a microphone, a speaker, a camera having a CMOS image sensor, and a display;
a processor;
a random-access memory;
a memory storing a data generation model obtained by deep learning on a neural network, and an emotion identification model;
a database; and
circuitry configured to:
receive, from the client terminal via the communication interface, sensor data comprising at least one of time-series numerical data obtained from a wearable device, voice data captured by the microphone, or image data captured by the camera;
store the sensor data in the database as structured tensor data;
analyze the sensor data using a time-series analysis model comprising at least one of a convolutional neural network, a recurrent neural network, or a Transformer-based model to generate an anomaly score and an event label;
estimate an emotion of a user by applying the emotion identification model to at least one of the voice data or the image data;
generate, using the data generation model, inference data comprising at least one of a recommendation label, a probability score, or natural language text, based on the anomaly score, the event label, and the estimated emotion;
adjust at least one of a format, a level of detail, or a length of the inference data based on the estimated emotion;
transmit, when the anomaly score exceeds a threshold, an alert to an external system via the communication interface; and
transmit the inference data to the client terminal via the communication interface, the inference data causing the client terminal to present the inference data to the user via at least one of the display or the speaker.

19. The system according to claim 18, wherein the data generation model comprises at least one of a text generation AI, an image generation AI, or a multimodal generation AI, and wherein the data generation model is a fine-tuned model configured to output inference results from prompts without instructions.

20. A method performed by circuitry of a system comprising a communication interface, a database, a data generation model obtained by deep learning on a neural network, and an emotion identification model, the method comprising:
receiving, from a client terminal via the communication interface and a packet-switched network, sensor data comprising at least one of time-series numerical data, voice data, or image data;
storing the sensor data in the database as structured tensor data;
analyzing the sensor data using a time-series analysis model comprising at least one of a convolutional neural network, a recurrent neural network, or a Transformer-based model to generate an anomaly score;
estimating an emotion of a user by applying the emotion identification model to the sensor data;
generating, using the data generation model, inference data comprising at least one of a recommendation label, a probability score, or natural language text, based on the anomaly score and the estimated emotion;
transmitting, when the anomaly score exceeds a threshold, an alert to an external system via the communication interface; and
transmitting the inference data to the client terminal via the communication interface and the packet-switched network, the inference data causing the client terminal to present the inference data to the user.
